(19) United States
(12) Patent Application Publication
(10) Pub. No.: US 2010/0322916 A1
(43) Pub. Date: Dec. 23, 2010

(54) DNA REPAIR POLYPEPTIDES AND METHODS OF DELIVERY AND USE

(76) Inventors: Amanda K. McCullough, Portland, OR (US); R. Stephen Lloyd, Portland, OR (US)

Correspondence Address: Klarquist Sparkman LLP, 121 SW Salmon Street, Suite 1600, Portland, OR, 97204, US

(21) Appl. No.: 12/865,213

(22) PCT Filed: Jan. 30, 2009
(86) PCT No.: PCT/US09/32710

§ 371 Date: Jul. 29, 2010

(51) Int. Cl. A61K 38/47; C12N 9/24; C07H 21/04; C12N 15/63; C12N 5/00; C12N 5/02

(52) U.S. Cl. 424/94.61; 435/200; 536/23.2; 435/320.1; 435/325; 435/375

(57) ABSTRACT

Described herein are pyrimidine dimer-specific glycosylase (PDG) polypeptides and methods of use for repair of damaged DNA. The PDG polypeptides comprise amino acid sequence from T4-PDG, CV-PDG or engineered mutants thereof. The PDG polypeptides further comprise a targeting sequence, such as a nuclear targeting sequence or a mitochondrial targeting sequence, and a protein transduction domain. The mutant PDG polypeptides described herein retain at least some catalytic activity while exhibiting reduced cytotoxicity in wild-type cells.

### PRIORITY

This claims the benefit of U.S. Provisional Application No. 61/024,845, filed Jan. 30, 2008, which is incorporated by reference herein in its entirety.

### STATEMENT OF GOVERNMENT SUPPORT

This invention was made in part with United States government support pursuant to grant ES04091, from the National Institute of Environmental Health Sciences, National Institutes of Health; the United States government has certain rights in the invention.

### FIELD

This disclosure concerns DNA repair polypeptides and methods of delivery and use thereof. In certain embodiments, this disclosure concerns methods of using pyrimidine dimer DNA glycosylases with enhanced DNA repair capacity and delivery, and decreased cytotoxicity for increasing the repair rate of damaged bases in a cell, such as a skin cell.

### BACKGROUND

The effects of excessive exposure to ultraviolet (UV) radiation include erythema, melanogenesis, photo-aging and wrinkling of the skin, cataract formation and the development of skin lesions, such as actinic keratoses, basal and squamous cell carcinomas. For example, exposure of human skin to the portion of the UV spectrum of sunlight that reaches the surface of the Earth (including a portion of the UVB and all of the UVA wavelengths) is a cause of nonmelanoma and melanoma skin cancer. The nonmelanoma cancers arise on sun-exposed areas of the body, while melanomas are more generally distributed across the body. Genetic changes in cells derived from these skin cancers reveal a very high frequency of tandem DNA mutations of CC to TT. This tandem mutation is strongly indicative of cis, syn cyclobutane pyrimidine dimers (cpds) and (6-4) photoproducts, two types of photoproducts produced by exposure of DNA to sunlight. Delay in the repair of cpds is a primary cause of UV-induced immunosuppression that can result in inefficient recognition and killing of emerging cancer cells. Alternatively, replication of unrepaired cpds can result in error-prone DNA synthesis, mutations and ultimately cancer. In order to remove this genetic damage, human cells utilize the nucleotide excision repair (NER) pathway, which removes a patch of damaged DNA by incising the damage-containing DNA strand both 5′ and 3′ to the damage. Polymerases and helicases act in conjunction to remove the patch and resynthesize new, undamaged DNA. A DNA ligase then completes repair by sealing the remaining break.

Further evidence implicating UV-induced DNA damage in the formation and progression of skin cancer is found in the human autosomal recessive disease, xeroderma pigmentosum. Individuals with xeroderma pigmentosum have a deficient NER pathway, rendering them exceptionally cancer prone, with their relative risk estimated to be 2000-fold greater than the average person.

Given the foregoing, it would be desirable to identify new treatments for repairing DNA lesions that result from UV light, for instance treatments for increasing the rate at which UV light-induced DNA damage is removed, thus decreasing premalignant and malignant lesions.

### SUMMARY

Described herein are pyrimidine dimer-specific glycosylase (PDG) polypeptides and methods of use for repair of damaged DNA. In one embodiment, the PDG polypeptide includes an amino acid sequence from T4-PDG or a mutant thereof. In another embodiment, the PDG polypeptide includes an amino acid sequence from CV-PDG or a mutant thereof. The mutant PDG polypeptides described herein retain at least some catalytic activity while exhibiting reduced cytotoxicity in wild-type cells.

Provided herein are isolated polypeptides including a PDG amino acid sequence, a targeting sequence and a protein transduction domain. In one embodiment, the targeting sequence is a nuclear localization sequence. In another embodiment, the targeting sequence is a mitochondrial localization sequence. In some embodiments, a protein transduction domain (e.g., a HIV transactivator of transcription (TAT) peptide) is included. Also provided are pharmaceutical compositions comprising a therapeutically effective amount of a PDG polypeptide described herein in a pharmaceutically acceptable carrier.

Further provided are isolated polynucleotides encoding the PDG polypeptides described herein, vectors including polynucleotides and cells including the polynucleotides.

Also provided are methods for increasing the repair rate of damaged bases in a cell and increasing the UV-resistance of a cell, comprising contacting a cell with a therapeutically effective concentration of an agent comprising an isolated PDG polypeptide described herein.

Further provided are methods of treating a skin disorder in a subject and treating UV-induced immunosuppression in a subject, including contacting the skin of the subject in need treatment with a therapeutically effective concentration of an agent comprising an isolated PDG polypeptide described herein.

The foregoing and other features will become more apparent from the following detailed description of several embodiments.

### Sequence Listing

The nucleic and amino acid sequences listed in the accompanying sequence listing are shown using standard letter abbreviations for nucleotide bases, and three letter code for amino acids, as defined in 37 C.F.R. 1.822. Only one strand of each nucleic acid sequence is shown, but the complementary strand is understood as included by any reference to the displayed strand. In the accompanying sequence listing:

SEQ ID NO: 1 is the amino acid sequence of wild-type T4-PDG.

SEQ ID NO: 2 is the amino acid sequence of R3Q T4-PDG.

SEQ ID NO: 3 is the amino acid sequence of R22Q T4-PDG.

SEQ ID NO: 4 is the amino acid sequence of R26Q T4-PDG.

SEQ ID NO: 5 is the amino acid sequence of R117Q T4-PDG.

SEQ ID NO: 6 is the amino acid sequence of wild-type CV-PDG.

SEQ ID NO: 7 is the amino acid sequence of R3Q CV-PDG.

SEQ ID NO: 8 is the amino acid sequence of R22Q CV-PDG.

SEQ ID NO: 9 is the amino acid sequence of R117Q CV-PDG.

SEQ ID NO: 10 is the amino acid sequence of R119Q CV-PDG.

SEQ ID NO: 11 is a nuclear localization amino acid sequence (NLS1).

SEQ ID NO: 12 is a nuclear localization amino acid sequence (NLS2).

SEQ ID NO: 13 is a mitochondrial targeting amino acid sequence.

SEQ ID NO: 14 is a mitochondrial targeting amino acid sequence.

SEQ ID NO: 15 is a mitochondrial targeting amino acid sequence derived from uracil DNA glycosylase mitochondrial targeting sequence (MTS29).

SEQ ID NO: 16 is the nucleotide sequence of forward primer CV-PDG Nde I.

SEQ ID NO: 17 is the nucleotide sequence of forward primer MLS35 CV-PDG Nde I.

SEQ ID NO: 18 is the nucleotide sequence of forward primer Delta 6 MLS35.

SEQ ID NO: 19 is the nucleotide sequence of forward primer NLS1 GFP pET22b

SEQ ID NO: 20 is the nucleotide sequence of forward primer NLS1 pET22b

SEQ ID NO: 21 is the nucleotide sequence of reverse primer CV-PDG.

SEQ ID NO: 22 is the nucleotide sequence of reverse primer CV-PDG NLS2.

SEQ ID NO: 23 is the nucleotide sequence of reverse primer CV-PDG GFP.

SEQ ID NO: 24 is the nucleotide sequence of reverse primer Delta 6 MLS35.

SEQ ID NO: 25 is the nucleotide sequence of reverse primer NLS1 pET22b.

SEQ ID NO: 26 is the nucleotide sequence of Chlorella virus isolate PBCV-1 pyrimidine dimer-specific glycosylase (CV-PDG; Genbank Accession No. AF128160).

SEQ ID NO: 27 is the nucleotide sequence of Bacteriophage T4 pyrimidine dimer-specific glycosylase (T4-PDG; nucleotides 1777-2193 of Genbank Accession No. X04567).

SEQ ID NO: 28 is amino acid sequence of Large T NLS.

SEQ ID NO: 29 is amino acid sequence of MA-NLS1 NLS.

SEQ ID NO: 30 is amino acid sequence of MA-NLS2 NLS.

SEQ ID NO: 31 is amino acid sequence of IN-NLS NLS.

SEQ ID NO: 32 is amino acid sequence of Vpr N NLS.

SEQ ID NO: 33 is amino acid sequence of Vpr C NLS.

SEQ ID NO: 34 is amino acid sequence of Rev NLS.

SEQ ID NO: 35 is amino acid sequence of H2B NLS.

SEQ ID NO: 36 is amino acid sequence of v-Jun NLS.

SEQ ID NO: 37 is amino acid sequence of nucleoplasmin NLS.

SEQ ID NO: 38 is amino acid sequence of NIN2 NLS.

SEQ ID NO: 39 is amino acid sequence of SWI5 NLS.

SEQ ID NO: 40 is the amino acid sequence of the HIV-1 TAT peptide.

SEQ ID NO: 41 is the amino acid sequence of a TAT peptide.

SEQ ID NO: 42 is the amino acid sequence of a TAT peptide.

SEQ ID NO: 43 is the amino acid sequence of a TAT peptide.

### DETAILED DESCRIPTION

### I. Introduction

Over a million new cases of skin cancer are diagnosed in the United States each year. The vast majority of nonmelanoma skin cancers, such as basal cell and squamous cell carcinomas, occur on portions of the body that are chronically exposed to sun. Following exposure to UV light, humans undergo a temporary, reversible immunosuppression (Norval et al., Photochemistry and Photobiology 84:19-28, 2008; Gruijl, Photochemistry and Photobiology 84:2-9, 2008). Previous data suggest that the molecular trigger for this immunosuppression is the persistence of the damaged DNA itself (Nishigori et al., Proc. Natl. Acad. Sci., U.S.A. 93:10354-10359, 1996; Wolf et al., J. Invest. Dermatol. 104:287-92, 1995).

UV-induced immunosuppression can result in inefficient recognition and killing of emerging cancer cells. In addition, replication of unrepaired cpds can result in error-prone DNA synthesis and mutations that lead to cancer. To remove these lesions, humans have only one, relatively inefficient mechanism of repair, the NER pathway.

However, human cells have components of an additional pathway for removing many types of DNA lesions, including cis-syn cyclobutane pyrimidine dimers, which arise from UV light, oxidative stress, alkylation damage and deamination, among others. This pathway is termed the base excision repair (BER) system. Although BER removes many lesions, humans lack the enzymes that initiate repair at sites of UV-induced damage. The first step in this pathway involves the recognition and removal of the damaged base by a class of enzymes called glycosylases. These enzymes break the glycosyl bond and a subset of these enzymes also possesses the ability to incise the phosphodiester backbone through a lyase reaction. Downstream of these reactions, the pathway requires the activities of an abasic site endonuclease, DNA polymerase(s) and DNA ligase. Therefore, the BER pathway is intact and robust, but humans lack the glycosylases required for initiating this pathway.

Glycosylases exist that can initiate repair at sites of UV-induced damage. The T4-PDG enzyme (also referred to as endonuclease V), produced by the deny gene of bacteriophage T4, catalyzes the first and rate limiting step in the removal of UV-induced DNA damage, namely, single strand incision of DNA at the site of damage. Other glycosylases having the ability to repair DNA damage have also been identified, and include the Micrococcus luteus ultraviolet N-glycosylase/apurinic/apyrimidinic (AP) lyase and the Paramecium bursaria chlorella virus-1 (PBCV-1) pyrimidine dimer-specific glycosylase (CV-PDG).

Provided herein are engineered PDG enzymes for activation of the BER pathway. In some embodiments, the engineered PDG polypeptide is a wild-type enzyme, such as T4-PDG or CV-PDG. In other embodiments, the engineered enzyme is a mutant PDG polypeptide, such as an enzyme exhibiting an alteration in catalytic activity, such as glycosylase activity and/or AP lyase activity, relative to a wild-type enzyme. In one embodiment, the wild-type or mutant PDG enzyme is fused to a targeting sequence, such as a nuclear targeting sequence or a mitochondrial targeting sequence. Such targeting sequences are described in detail herein.

In order to deliver the PDG polypeptides to cells in the epidermis and dermis, previously it has been necessary to encapsulate them into liposomes and following topical administration, these liposomes will fuse with cell membranes and deliver the aqueous-soluble repair enzymes to the cells. This strategy has limitations concerning stability of the liposome and activity of the enzymes. Therefore, the present disclosure describes an alternative mechanism of epidermal and dermal delivery of these enzymes without the need of liposomes. It is believed that fusion of the protein transduction domain (PTD) of TAT to the carboxy-terminal region of PDGs facilitates the efficient delivery of active DNA repair proteins to the skin. This disclosure provides a mechanism for the delivery of proteins into the epidermal and dermal skin layers, and will have the capacity to reduce or eliminate skin cancers.

### II. Abbreviations and Terms

AP: apurinic/apyrimidinic
BER: base excision repair
BSA: bovine serum albumin
CMV: cytomegalovirus
CPD: cyclobutane pyrimidine dimers
cpds: cis, syn cyclobutane pyrimidine dimers
CV-PDG: chlorella virus encoded pyrimidine dimer glycosylase
DNA: deoxyribonucleic acid
EGFP: enhanced green fluorescent protein
ELISA: enzyme linked immunosorbent assay
FBS: fetal bovine serum
HIV: human immunodeficiency virus
IPTG: isopropyl-1-thio-β-D-galactoside
MLS: mitochondrial localization sequence
MTS: mitochondrial targeting sequence
NER: nucleotide excision repair
NLS: nuclear localization sequence
PBCV: Paramecium bursaria chlorella virus
PBS: phosphate buffered saline
PCR: polymerase chain reaction
PDG: pyrimidine dimer glycosylase
PTD: protein transduction domain
T4-PDG: bacteriophage T4 pyrimidine dimer glycosylase
TAT: trans activator of transcription
tRNA: transfer ribonucleic acid
UV: ultraviolet
WT: wild-type
XPA: Xeroderma pigmentosum cells of complementation group A

Unless otherwise noted, technical terms are used according to conventional usage. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

In order to facilitate review of the various embodiments, the following explanations of specific terms are provided:

Actinic keratosis: A precancerous skin condition caused by overexposure to the sun. Actinic keratoses are small (usually less than one-fourth inch) rough spots that may be pink-red or flesh-colored. Usually they develop on sun-exposed areas of the skin, such as the face, ears, back of the hands, and arms, although they can arise on other sun-exposed areas of the skin. Actinic keratoses are slow growing. They usually do not cause any symptoms or signs other than patches on the skin. It is possible, but not common, for actinic keratoses to turn into squamous cell cancer. They also frequently go away on their own but may come back.

Administer: To provide or give a subject an agent, such as one of the disclosed polypeptides, by any effective route. Administration can be systemic or local. Exemplary routes of administration include, but are not limited to, oral, injection (such as subcutaneous, intramuscular, intradermal, intraperitoneal and intravenous), sublingual, rectal, transdermal (e.g., topical), intranasal, vaginal and inhalation routes. For example, if the chosen route is intravenous, the composition is administered by introducing the composition into a vein of the subject. Alternatively, if the chosen route is intramuscular, the composition is administered by introducing the composition in to a muscle. In particular examples, agents (such as those including one of the disclosed polypeptides) are administered to a subject having or at risk of UV-induced DNA damage, such as that associated with skin cancer.

Agent: Any protein, nucleic acid molecule, compound, small molecule, organic compound, inorganic compound, or other molecule of interest. Agent can include a therapeutic agent, a diagnostic agent or a pharmaceutical agent. A therapeutic or pharmaceutical agent is one that alone or together with an additional agent (such as an antineoplastic agent, such as Etoposide, Doxorubicin, methotrexate, and Vincristine) induces the desired response (such as inducing a therapeutic or prophylactic effect when administered to a subject). In an example, an agent includes one of the disclosed polypeptides. In a particular example, an agent specifically increases the repair rate of damaged bases in a cell, thereby reducing or inhibiting the tumor, such as a skin tumor.

Apurinic/apyrimidinic lyase activity (AP lyase activity): The ability of a polypeptide to catalyze a β-elimination reaction on an abasic site containing DNA, resulting in an α,β-unsaturated aldehyde. A polypeptide having pyrimidine glycosylase activity and AP lyase activity is referred to herein as a “pyrimidine glycosylase/AP lyase,” and has “pyrimidine glycosylase/AP lyase activity.” A polypeptide having pyrimidine glycosylase/AP lyase activity is referred to as a “pyrimidine dimer specific DNA glycosylase/Alyase.”

Whether a polypeptide has pyrimidine glycosylase/AP lyase activity can be determined by measuring the ability of the polypeptide to incise a target polynucleotide containing damaged bases in the presence of a buffer. The target polynucleotide contains damaged bases, such as UV radiation-induced pyrimidine dimers. As one example, the target polynucleotide is present at a concentration of from about 0.1 nM to about 10 nM. The putative glycosylase/AP lyase is present at a concentration of from about 0.01 nM to about 100 nM. Buffers in which a glycosylase/AP lyase is active are suitable for the assay. For example, one such buffer has a pH of approximately 6.5 to 7.5 and includes approximately 25 mM NaH₂PO₄; 10-125 mM NaCl; 1-10 mM EDTA; and 0.01-1.0 mg/mL bovine serum albumin (BSA). The temperature of the assay can be about 37° C. The assay can be carried out for at least about 10 seconds to no greater than about 8 hours. A polypeptide having pyrimidine glycosylase/AP lyase activity will cause the mobility of the target polynucleotide to change relative to the polynucleotide that has not been exposed to the polypeptide. The polypeptide can be present in a crude cellular extract, isolated or purified. Since polypeptides identified in this assay as having pyrimidine glycosylase/AP lyase activity function on UV-irradiated DNAs, these polypeptides identify cyclobutane pyrimidine dimers, and are likely to be active on other UV-induced photoproducts including FapyA and FapyG.

Cancer: A malignant tumor characterized by abnormal or uncontrolled cell growth. Other features often associated with cancer include metastasis, interference with the normal functioning of neighboring cells, release of cytokines or other secretory products at abnormal levels and suppression or aggravation of inflammatory or immunological response, invasion of surrounding or distant tissues or organs, such as lymph nodes, etc. “Metastatic disease” refers to cancer cells that have left the original tumor site and migrate to other parts of the body for example via the bloodstream or lymph system.

In one example, an agent including one of the disclosed polypeptides is administered to a subject to prevent or treat skin cancer. Skin cancer is a malignant growth on the skin which can have many causes. Skin cancer generally develops in the epidermis (the outermost layer of skin), so a tumor is usually clearly visible. This makes most nonmelanoma skin cancers detectable in the early stages. Skin cancer represents the most commonly diagnosed malignancy, surpassing lung, breast, colorectal and prostate cancer.

The most common type of skin cancer is nonmelanoma skin cancer. Nonmelanoma skin cancers include all skin cancers except malignant melanoma (cancer that develop from melanocytes, the pigment-producing cells of the skin). There are many types of nonmelanoma skin cancers. Two common types of nonmelanoma skin cancer are basal cell carcinoma and squamous cell carcinoma. These two types of skin cancer are also known as keratinocyte carcinomas.

Basal cell carcinoma begins in the lowest layer of the epidermis, called the basal cell layer. About 70% to 80% of all skin cancers in men and 80% to 90% in women are basal cell carcinomas. They usually develop on sun-exposed areas, especially the head and neck. Basal cell carcinoma is slow growing. It is highly unusual for a basal cell cancer to spread to lymph nodes or to distant parts of the body. However, if a basal cell cancer is left untreated, it can grow into nearby areas and invade the bone or other tissues beneath the skin. After treatment, basal cell carcinoma can recur in the same place on the skin. Also, new basal cell cancers can start elsewhere on the skin. Within 5 years of being diagnosed with one basal cell cancer, 35% to 50% of people develop a new skin cancer.

Squamous cell carcinomas account for about 10% to 30% of all skin cancers. They commonly appear on sun-exposed areas of the body such as the face, ear, neck, lip, and back of the hands Squamous cell carcinomas can also develop in scars or skin ulcers elsewhere. These carcinomas are generally more aggressive than basal cell cancers. Squamous cell carcinomas can sometimes start in actinic keratoses. Squamous cell carcinoma in situ (also called Bowen disease) is the earliest form of squamous cell skin cancer and involves cells that are within the epidermis and have not invaded the dermis.

Less common types of nonmelanoma skin cancer include Kaposi sarcoma, cutaneous lymphoma, skin adnexal tumors and various types of sarcomas and Merkel cell carcinoma. Together, these types of nonmelanoma skin cancer account for less than 1% of nonmelanoma skin cancers.

The most lethal type of skin cancer is melanoma. Melanoma (also known as malignant melanoma or cutaneous melanoma) is a cancer that begins in the melanocytes. Because most melanoma cells still produce melanin, melanoma tumors are usually brown or black. This form of skin cancer can be fatal if not treated early.

Chemotherapeutic agent: An agent with therapeutic usefulness in the treatment of diseases characterized by abnormal cell growth (e.g., an anti-neoplastic agent). Such diseases include tumors, neoplasms, and cancer, as well as diseases characterized by hyperplastic growth such as psoriasis. In one embodiment, a chemotherapeutic agent is an agent of use in treating neoplasms such as solid tumors. In one embodiment, a chemotherapeutic agent is radioactive molecule. One of skill in the art can readily identify a chemotherapeutic agent (for instance, see Slapak and Kufe, Principles of Cancer Therapy, Chapter 86 in Harrison's Principles of Internal Medicine, 14th edition; Perry et al., Chemotherapy, Ch. 17 in Abeloff, Clinical Oncology 2^{nd} ed., © 2000 Churchill Livingstone, Inc; Baltzer L, Berkery R (eds): Oncology Pocket Guide to Chemotherapy, 2nd ed. St. Louis, Mosby-Year Book, 1995; Fischer D S, Knobf M F, Durivage H J (eds): The Cancer Chemotherapy Handbook, 4th ed. St. Louis, Mosby-Year Book, 1993).

Contacting: Placement in direct physical association, including both a solid and liquid form. Contacting can occur in vitro with isolated cells or in vivo by administering to a subject. In an example, DNA repair activity is increased by contacting or exposing a cell, such as a tumor cell, with a therapeutically effective concentration of an agent, including one of the disclosed polypeptides.

Damaged base: Structural deviations in nucleoside-5′-monophosphates present in the genomic DNA of a eukaryotic cell. One type of structural deviation is a covalent joining of the adjacent pyrimidines through the formation of a cyclobutane ring structure at the C5 and C6 positions. Another type of structural deviation is an imidazole ring fragmentation of a purine (either adenine or guanine). The location of such structural deviations in a cell's genomic DNA is referred to as a “lesion.” Damaged bases can arise from, for example, UV radiation, ionizing radiation, oxidative stress, alkylation damage or deamination. Examples of lesions include cis-syn and trans-syn II cyclobutane pyrimidine dimers, FapyA and FapyG (Lloyd, Mutat. Res. 408:159-170, 1998; Lloyd, Progress in Nucleic Acid Research and Molecular Biology 62:155-175, 1999).

Disease: An abnormal condition of an organism that impairs bodily functions.

DNA repair: A collection of processes by which a cell identifies and corrects damage to the DNA molecules that encode its genome. In human cells, both normal metabolic activities and environmental factors such as UV light can cause DNA damage, resulting in as many as 1 million individual molecular lesions per cell per day. Many of these lesions cause structural damage to the DNA molecule and can alter or eliminate the cell's ability to transcribe the gene that the affected DNA encodes. Other lesions induce potentially harmful mutations in the cell's genome. Consequently, the DNA repair process must be constantly active so it can respond rapidly to any damage in the DNA structure.

The rate of DNA repair is dependent on many factors, including the cell type, the age of the cell, and the extracellular environment. A cell that has accumulated a large amount of DNA damage, or one that no longer effectively repairs damage incurred to its DNA, can enter one of three possible states: an irreversible state of dormancy, known as senescence; apoptosis or programmed cell death or unregulated cell division, which can lead to the formation of a tumor that is cancerous.

In one example, a cell in need of DNA repair is contacted with a therapeutically effective concentration of an agent including one of the disclosed PDG polypeptides to increase the repair rate of damaged DNA in the cell compared to an untreated cell. For example, the repair rate can be increased by at least 10%, such as by at least 20%, at least 30%, at least 40%, at least 50%, at least 60%, at least 70%, at least 80%, or at least 90%.

Fusion Protein: A protein generated by expression of a nucleic acid sequence engineered from nucleic acid sequences encoding at least a portion of two or more different (heterologous) proteins. To create a fusion protein, the nucleic acid sequences must be in the same reading frame and contain no internal stop codons. Methods of preparing fusion proteins are well known in the art. Provided herein are fusion proteins including at least a portion of a PDG polypeptide sequence. In one embodiment, the fusion protein includes a targeting sequence, such as a NLS or MLS. In another embodiment, the PDG fusion protein includes a protein transduction domain (PTD), such as TAT. In a preferred embodiment, the fusion protein includes a PDG polypeptide sequence, a targeting sequence and a PTD.

Genomic DNA: DNA present in the nucleus of a cell.

Isolated polypeptide or polynucleotide: A polypeptide or polynucleotide that has been either removed from its natural environment, produced using recombinant techniques or chemically or enzymatically synthesized.

Keratinocyte: The major cell type of the epidermis, making up about 90% of epidermal cells. The epidermis is divided into four or five layers (depending on the type of skin) based on keratinocyte morphology: stratum basal (at the junction with the dermis); stratum spinosum; stratum granulosum; stratum lucidum (only present in thick skin, such as the palms of the hand and soles of the feet) and stratum corneum.

Keratinocytes originate in the basal layer from the division of keratinocyte stem cells. They are pushed up through the layers of the epidermis, undergoing gradual differentiation until they reach the stratum corneum where they form a layer of enucleated, flattened, highly keratinized cells called squamous cells. This layer forms an effective barrier to the entry of foreign matter and infectious agents into the body and minimizes moisture loss. Keratinocytes are shed and replaced continuously from the stratum corneum. The time of transit from basal layer to shedding is approximately one month. Although that approximate time frame can be accelerated in conditions of keratinocyte hyperproliferation, such as psoriasis.

Malignant cells: Cells which have the properties of anaplasia, invasion and metastasis.

Mitochondrial localization or targeting sequence (MLS): A targeting sequence that causes the polypeptide to which it is fused to migrate and be incorporated into the mitochondria. MLSs are generally located at the N-terminal portion of the polypeptide. For PDGs, the MLS must be removed during the import process, regenerating the active site. In one example, the MLS is present at the amino terminal end of a PDG polypeptide. “Mitochondrial localization sequence” and “mitochondrial localization signal” are both referred to herein as “MLS” and can be used interchangeably with “mitochondrial targeting sequence (MTS)” and “mitochondrial targeting signal (MTS).” The amino acid sequences of examples of MLSs that can be used in the present disclosure include those provided in SEQ ID NOs: 13, 14 and 15; however, additional MLSs are known in the art and can be used with the PDG polypeptides described herein.

Mutant: As used herein, a “mutant” PDG refers to a T4-PDG or CV-PDG enzyme comprising one or more genetic mutations resulting in at least one amino acid change (also referred to as a “substitution”). In some embodiments, PDG mutants exhibit an alteration in catalytic activity, such as an alteration in glycosylase activity and/or AP lyase activity. PDG mutants are referred to herein by their mutation. For example, the T4-PDG mutant “R3Q” refers to the mutant wherein the arginine residue (R) is replaced by a glutamine (Q).

Neoplasm: Abnormal growth of cells, for example a tumor.

Normal cells: Non-diseased cells, such as non-tumor, non-malignant cells.

Nuclear localization or targeting sequence (NLS): A targeting sequence that causes the polypeptide to which it is fused to migrate to the nucleus. An NLS can be present in any location in a polypeptide provided that the NLS does not inhibit or interfere with PDG activity of the polypeptide after the PDG is delivered to the nucleus. In one example, the NLS is present at the carboxy terminal end of a PDG polypeptide. A NLS is also referred to as a “nuclear localization signal.” The amino acid sequences of examples of NLSs that can be used in the present disclosure include those provided in SEQ ID NOs: 11, 12 and 28-39; however, additional NLSs are known in the art and can be used with the PDG polypeptides described herein.

ORF (open reading frame): A series of nucleotide triplets (codons) coding for amino acids without any termination codons. These sequences are usually translatable into a peptide.

Operably linked: A first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein-coding regions, in the same reading frame.

PDG polypeptide: A polypeptide comprising at least a portion of a pyrimidine dimer-specific DNA glycosylase. In one embodiment, the PDG polypeptide comprises amino acid sequence from T4-PDG. In another embodiment, the PDG polypeptide comprises amino acid sequence from CV-PDG. The PDG polypeptides disclosed herein can optionally comprise a targeting sequence, such as a NLS or MLS and/or a PTD, such as the TAT peptide. Thus, as used herein, a “PDG polypeptide” refers to PDG polypeptides alone or when fused to other protein domains, such as targeting sequences, PTDs, or other domains such as domains to facilitate protein purification.

Pharmaceutical agent: A chemical compound or other composition capable of inducing a desired therapeutic or prophylactic effect when properly administered to a subject. A pharmaceutical agent also includes a “drug.”

Pharmaceutically Acceptable Vehicles: The pharmaceutically acceptable carriers (vehicles) useful in this disclosure are conventional. Remington's Pharmaceutical Sciences, by E. W. Martin, Mack Publishing Co., Easton, Pa., 15th Edition (1975), describes compositions and formulations suitable for pharmaceutical delivery of one or more therapeutic compounds or molecules, such as one or more T4-PDG nucleic acid molecules or proteins, and additional pharmaceutical agents.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (for example, powder, pill, tablet, or capsule forms), conventional non-toxic solid carriers can include, for example, pharmaceutical grades of mannitol, lactose, starch, or magnesium stearate. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

Polypeptide: A polymer in which the monomers are amino acid residues which are joined together through amide bonds. When the amino acids are alpha-amino acids, either the L-optical isomer or the D-optical isomer can be used. The terms “polypeptide” or “protein” as used herein are intended to encompass any amino acid sequence and include modified sequences such as glycoproteins. The term “polypeptide” is specifically intended to cover naturally occurring proteins, as well as those which are recombinantly or synthetically produced.

The term “residue” or “amino acid residue” includes reference to an amino acid that is incorporated into a protein, polypeptide, or peptide.

Conservative amino acid substitutions are those substitutions that, when made, least interfere with the properties of the original protein, that is, the structure and especially the function of the protein is conserved and not significantly changed by such substitutions. Examples of conservative substitutions are shown below.

| Original Residue | Conservative Substitutions |
|---|---|
| Ala | Ser |
| Arg | Lys |
| Asn | Gln, His |
| Asp | Glu |
| Cys | Ser |
| Gln | Asn |
| Glu | Asp |
| His | Asn; Gln |
| Ile | Leu, Val |
| Leu | Ile; Val |
| Lys | Arg; Gln; Glu |
| Met | Leu; Ile |
| Phe | Met; Leu; Tyr |
| Ser | Thr |
| Thr | Ser |
| Trp | Tyr |
| Tyr | Trp; Phe |
| Val | Ile; Leu |

Conservative substitutions generally maintain (a) the structure of the polypeptide backbone in the area of the substitution, for example, as a sheet or helical conformation, (b) the charge or hydrophobicity of the molecule at the target site, or (c) the bulk of the side chain.

The substitutions which in general are expected to produce the greatest changes in protein properties will be non-conservative, for instance changes in which (a) a hydrophilic residue, for example, seryl or threonyl, is substituted for (or by) a hydrophobic residue, for example, leucyl, isoleucyl, phenylalanyl, valyl or alanyl; (b) a cysteine or proline is substituted for (or by) any other residue; (c) a residue having an electropositive side chain, for example, lysyl, arginyl, or histadyl, is substituted for (or by) an electronegative residue, for example, glutamyl or aspartyl; or (d) a residue having a bulky side chain, for example, phenylalanine, is substituted for (or by) one not having a side chain, for example, glycine.

Preventing, treating or ameliorating a disease: “Preventing” a disease refers to inhibiting the full development of a disease. “Treating” refers to a therapeutic intervention that ameliorates a sign or symptom of a disease or pathological condition after it has begun to develop. “Ameliorating” refers to the reduction in the number or severity of signs or symptoms of a disease.

Promoter: A promoter is an array of nucleic acid control sequences which direct transcription of a nucleic acid. A promoter includes necessary nucleic acid sequences near the start site of transcription. A promoter also optionally includes distal enhancer or repressor elements. A “constitutive promoter” is a promoter that is continuously active and is not subject to regulation by external signals or molecules. In contrast, the activity of an “inducible promoter” is regulated by an external signal or molecule (for example, a transcription factor).

Protein transduction domain (PTD): A polypeptide, polynucleotide, carbohydrate, or organic or inorganic compound that facilitates traversing a lipid bilayer, micelle, cell membrane, organelle membrane or vesicle membrane. In one example, the protein transduction domain is a HIV transactivator of transcription (TAT) protein which facilitates the introduction of one of the disclosed PDG polypeptides into the epidermal and dermal skin layers. PTDs can be naturally occurring or synthetically produced.

Psoriasis: A chronic disorder which affects the skin. Psoriasis commonly causes red scaly patches to appear on the skin. The scaly patches caused by psoriasis, called psoriatic plaques, are areas of inflammation and excessive skin production. Psoriasis varies in severity from minor localized patches to complete body coverage. Psoriasis can also cause inflammation of the joints, which is known as psoriatic arthritis. In an example, an agent including one of the disclosed PDG polypeptides is administered to a subject, such as by a topical lotion, to treat psoriasis or a sign or symptom associated with psoriasis.

Purified: The term purified does not require absolute purity; rather, it is intended as a relative term. Thus, for example, a purified protein preparation is one in which the protein referred to is more pure than the protein in its natural environment within a cell.

In certain embodiments, the term “substantially purified” refers to a peptide, protein, or other active compound that has been isolated from a cell, cell culture medium, or other crude preparation and subjected to fractionation to remove various components of the initial preparation, such as proteins, cellular debris, and other components. Such purified preparations can include materials in covalent association with the active agent, such as glycoside residues or materials admixed or conjugated with the active agent, which may be desired to yield a modified derivative or analog of the active agent or produce a combinatorial therapeutic formulation, conjugate, fusion protein or the like. The term purified thus includes such desired products as peptide and protein analogs or mimetics or other biologically active compounds wherein additional compounds or moieties are bound to the active agent in order to allow for the attachment of other compounds and/or provide for formulations useful in therapeutic treatment or diagnostic procedures.

Generally, substantially purified peptides, proteins, or other active compounds include more than 80% of all macromolecular species present in a preparation prior to admixture or formulation of the respective compound with additional ingredients in a complete pharmaceutical formulation for therapeutic administration. Additional ingredients can include a pharmaceutical carrier, excipient, buffer, absorption enhancing agent, stabilizer, preservative, adjuvant or other like co-ingredients. More typically, the peptide, protein or other active compound is purified to represent greater than 90%, often greater than 95% of all macromolecular species present in a purified preparation prior to admixture with other formulation ingredients. In other cases, the purified preparation may be essentially homogeneous, wherein other macromolecular species are less than 1%.

Pyrimidine dimer glycosylase (PDG): A polypeptide that recognizes the presence of two consecutive damaged bases in a polynucleotide and catalyzes the breakage of the glycosyl bond between the 5′ base and the DNA sugar-phosphate backbone. A polypeptide that recognizes the presence of two consecutive damaged pyrimidine bases and catalyzes the breakage of such a bond has “glycosylase activity.” Whether a polypeptide has pyrimidine dimer glycosylase activity can be determined by measuring the ability of the polypeptide to cleave the glycosyl bond of the 5′ pyrimidine of a cyclobutane pyrimidine dimer in DNA. Such methods are well known to the art. A polypeptide having pyrimidine dimer glycosylase activity is often referred to as a pyrimidine dimer-specific DNA glycosylase.

Recombinant Nucleic Acid: A nucleic acid sequence that is not naturally occurring or has a sequence that is made by an artificial combination of two otherwise separated segments of sequence. This artificial combination is often accomplished by chemical synthesis or, more commonly, by the artificial manipulation of isolated segments of nucleic acids, for example, by genetic engineering techniques such as those described in Sambrook et al. (ed.), Molecular Cloning: A Laboratory Manual, 2^{nd} ed., vol. 1-3, Cold Spring Harbor Laboratory Press, Cold Spring Harbor, N.Y., 1989. The term recombinant includes nucleic acids that have been altered solely by addition, substitution, or deletion of a portion of the nucleic acid.

Reporter gene: A reporter gene is a gene operably linked to another gene or nucleic acid sequence of interest (such as a promoter sequence). Reporter genes are used to determine whether the gene or nucleic acid of interest is expressed in a cell or has been activated in a cell. Reporter genes typically have easily identifiable characteristics, such as fluorescence, or easily assayed products, such as an enzyme. Reporter genes can also confer antibiotic resistance to a host cell.

Sequence identity: The similarity between two nucleic acid sequences or between two amino acid sequences is expressed in terms of the level of sequence identity shared between the sequences. Sequence identity is typically expressed in terms of percentage identity; the higher the percentage, the more similar the two sequences.

Methods for aligning sequences for comparison are well known in the art. Various programs and alignment algorithms are described in: Smith and Waterman, Adv. Appl. Math. 2:482, 1981; Needleman and Wunsch, J. Mol. Biol. 48:443, 1970; Pearson and Lipman, Proc. Natl. Acad. Sci. USA 85:2444, 1988; Higgins and Sharp, Gene 73:237-244, 1988; Higgins and Sharp, CABIOS 5:151-153, 1989; Corpet et al., Nucleic Acids Research 16:10881-10890, 1988; Huang, et al., Computer Applications in the Biosciences 8:155-165, 1992; Pearson et al., Methods in Molecular Biology 24:307-331, 1994; Tatiana et al., (1999), FEMS Microbiol. Lett., 174:247-250, 1999. Altschul et al. present a detailed consideration of sequence-alignment methods and homology calculations (J. Mol. Biol. 215:403-410, 1990).

The National Center for Biotechnology Information (NCBI) Basic Local Alignment Search Tool (BLAST™, Altschul et al. J. Mol. Biol. 215:403-410, 1990) is available from several sources, including the National Center for Biotechnology Information (NCBI, Bethesda, Md.) and on the Internet, for use in connection with the sequence-analysis programs blastp, blastn, blastx, tblastn and tblastx. A description of how to determine sequence identity using this program is available on the internet under the help section for BLAST™.

For comparisons of amino acid sequences of greater than about 30 amino acids, the “Blast 2 sequences” function of the BLAST™ (Blastp) program is employed using the default BLOSUM62 matrix set to default parameters (cost to open a gap [default=5]; cost to extend a gap [default=2]; penalty for a mismatch [default=−3]; reward for a match [default=1]; expectation value (E) [default=10.0]; word size [default=3]; number of one-line descriptions (V) [default=100]; number of alignments to show (B) [default=100]). When aligning short peptides (fewer than around 30 amino acids), the alignment should be performed using the Blast 2 sequences function, employing the PAM30 matrix set to default parameters (open gap 9, extension gap 1 penalties). Proteins (or nucleic acids) with even greater similarity to the reference sequences will show increasing percentage identities when assessed by this method, such as at least 50%, at least 60%, at least 70%, at least 80%, at least 85%, at least 90%, or at least 95% sequence identity.

For comparisons of nucleic acid sequences, the “Blast 2 sequences” function of the BLAST™ (Blastn) program is employed using the default BLOSUM62 matrix set to default parameters (cost to open a gap [default=11]; cost to extend a gap [default=1]; expectation value (E) [default=10.0]; word size [default=11]; number of one-line descriptions (V) [default=100]; number of alignments to show (B) [default=100]). Nucleic acid sequences with even greater similarity to the reference sequences will show increasing percentage identities when assessed by this method, such as at least 60%, at least 70%, at least 75%, at least 80%, at least 85%, at least 90%, at least 95%, or at least 98% sequence identity.

An alternative indication that two nucleic acid molecules are closely related is that the two molecules hybridize to each other under stringent conditions (see “Hybridization” above).

Nucleic acid sequences that do not show a high degree of identity can nevertheless encode similar amino acid sequences, due to the degeneracy of the genetic code. It is understood that changes in nucleic acid sequence can be made using this degeneracy to produce multiple nucleic acid molecules that all encode substantially the same protein.

Subject: Living multi-cellular vertebrate organisms, a category that includes human and non-human mammals (such as laboratory or veterinary subjects). In an example, a subject is a human. In an additional example, a subject is selected that is in need of preventing or inhibiting a tumor, such as a skin cancer. For example, the subject is either at risk of developing a tumor or has a tumor, such as a skin cancer, in need of treatment.

Therapeutically effective amount: An amount of an agent (such as an agent that includes one of the disclosed PDG polypeptides), that alone, or together with one or more additional therapeutic agents (such antineoplastic agents), induces the desired response, such as prevention or treatment of a tumor, such as skin cancer. In one example, it is an amount of an agent including one of the disclosed PDG polypeptides needed to prevent or delay the development of a tumor, prevent or delay the metastasis of a tumor, cause regression of an existing tumor, or treat one or more signs or symptoms associated with a tumor, in a subject. Ideally, a therapeutically effective amount provides a therapeutic effect without causing a substantial cytotoxic effect in the subject. The preparations disclosed herein are administered in therapeutically effective amounts.

In one example, a desired response is to increase DNA repair enzymes, thereby decreasing the size, volume, or metastasis of a tumor, such as skin cancer. For example, the agent can decrease the size, volume, or metastasis of a tumor by a desired amount, for example by at least 5%, at least 10%, at least 15%, at least 20%, at least 25%, at least 30%, at least 50%, at least 75%, or even at least 90%, as compared to a response in the absence of the agent.

The effective amount of an agent that includes one of the disclosed polypeptides, that is administered to a human or veterinary subject will vary depending upon a number of factors associated with that subject, for example the overall health of the subject. An effective amount of an agent can be determined by varying the dosage of the product and measuring the resulting therapeutic response, such as the regression of a tumor. Effective amounts also can be determined through various in vitro, in vivo or in situ immunoassays. The disclosed agents can be administered in a single dose, or in several doses, as needed to obtain the desired response. However, the effective amount of can be dependent on the source applied, the subject being treated, the severity and type of the condition being treated, and the manner of administration.

In particular examples, a therapeutically effective dose of an agent including a disclosed PDG polypeptide is at least 1 μg daily (such as 1-100 μg or 5-50 μg) if administered via injection, or at least 1 mg daily if administered topically (such as 1-100 mg or 5-50 mg). In particular examples, such daily dosages are administered in one or more divided doses (such as 2, 3, or 4 doses) or in a single formulation.

The disclosed agents can be administered alone, in the presence of a pharmaceutically acceptable carrier, in the presence of other therapeutic agents (such as other anti-neoplastic agents), or both.

Treated cell: A cell that has been contacted with a desired agent in an amount and under conditions sufficient for the desired response. In one example, a treated cell is a cell that has been exposed to at least one of the disclosed PDG polypeptides under conditions sufficient to increase the rate of DNA repair. In another example, a “treated cell” is a cell exposed to UV light.

Treating or treatment: Refers to a therapeutic intervention that ameliorates a sign or symptom of a disease or pathological condition related to a disease (such as xeroderma pigmentosum, psoriasis, or a tumor, for example, cancer). Treatment can also induce remission or cure such condition. In particular examples, treatment includes inhibiting a tumor, for example by inhibiting the full development of a tumor, such as preventing development of a metastasis or the development of a primary tumor. Inhibition does not require a total absence of a tumor. In other examples, treatment includes inhibiting or reducing skin cancer.

Reducing or suppressing a sign or symptom associated with a disease (xeroderma pigmentosum, psoriasis, or a tumor, for example, cancer) can be evidenced, for example, by a delayed onset of clinical symptoms of the disease in a susceptible subject (such as a subject having a tumor which has not yet metastasized), a reduction in severity of some or all clinical symptoms of the disease, a slower progression of the disease (for example by prolonging the life of a subject having the disease), a reduction in the number of relapses of the disease, an improvement in the overall health or well-being of the subject, or by other parameters well known in the art that are specific to the particular disease.

Tumor: A neoplasm that may be either benign or malignant. In an example, a tumor is a malignant tumor, such as skin cancer. In another example, a tumor is a benign skin tumor, such as a keratoacanthoma (a common benign growth that is found on sun-exposed skin).

Under conditions sufficient for: A phrase that is used to describe any environment that permits the desired activity. In one example, includes administering a therapeutically effective amount of a composition that includes a disclosed PDG polypeptide, sufficient to allow the desired activity. In particular examples, the desired activity is inhibiting or preventing a tumor, such as skin cancer.

Unit dose: A physically discrete unit containing a predetermined quantity of an active material calculated to individually or collectively produce a desired effect, such as a therapeutic effect. A single unit dose or a plurality of unit doses can be used to provide the desired effect, such as treatment of a disease, for example a recurring skin tumor (e.g., skin cancer), xeroderma pigmentosum, or psoriasis.

Untreated cell: A cell that has not been contacted with a desired agent, such as a test agent. In an example, an untreated cell is a cell that receives the vehicle without the desired agent. In another example, an untreated cell is one that is not exposed to UV light.

Vector: A nucleic acid molecule as introduced into a host cell, thereby producing a transformed host cell. A vector can include nucleic acid sequences that permit it to replicate in the host cell, such as an origin of replication. A vector can also include one or more selectable marker genes and other genetic elements. An insertional vector is capable of inserting itself into a host nucleic acid. An expression vector is a vector that contains the necessary regulatory sequences to allow transcription and translation of inserted gene or genes.

Xeroderma pigmentosum: An autosomal recessive genetic disorder of DNA repair. This condition leads to multiple basaliomas and other skin malignancies at a young age. In severe cases, it is necessary to avoid sunlight completely. The most common defect in xeroderma pigmentosum is a genetic defect whereby NER enzymes are mutated, leading to a reduction in or elimination of NER. Unrepaired damage can lead to mutations, altering the information of the DNA. Subjects with xeroderma pigmentosum have a predisposition for cancer.

Unless otherwise explained, all technical and scientific terms used herein have the same meaning as commonly understood by one of ordinary skill in the art to which this disclosure belongs. The singular terms “a,” “an,” and “the” include plural referents unless context clearly indicates otherwise. Similarly, the word “or” is intended to include “and” unless the context clearly indicates otherwise. Hence “comprising A or B” means including A, or B, or A and B. It is further to be understood that all base sizes or amino acid sizes, and all molecular weight or molecular mass values, given for nucleic acids or polypeptides are approximate, and are provided for description.

Although methods and materials similar or equivalent to those described herein can be used in the practice or testing of the present disclosure, suitable methods and materials are described below. In case of conflict, the present specification, including explanations of terms, will control. In addition, the materials, methods, and examples are illustrative only and not intended to be limiting.

### III. Overview of Several Embodiments

The present disclosure provides compositions and methods for activation of the base-excision repair (BER) pathway in mammalian cells. As described herein, to activate this pathway, mammalian cells require enzymes with glycosylase and/or AP lyase activity. Thus, provided herein are polypeptides with PDG activity and polynucleotides encoding the PDG polypeptides. In some aspects of the disclosure, the PDG polypeptides have reduced catalytic activity, such as pyrimidine glycosylase activity and/or AP lyase activity, but also exhibit reduced cytotoxicity in wild-type cells, resulting in increased survival of wild-type cells when treated with the PDG polypeptides. Typically, the PDG is T4-PDG, CV-PDG, or a mutant thereof.

In one embodiment, the PDG polypeptides include an amino acid sequence from a mutant PDG polypeptide, such as a mutant PDG polypeptide having the sequence set forth as SEQ ID NO: 2, SEQ ID NO: 3, SEQ ID NO: 4, SEQ ID NO: 5, SEQ ID NO: 7, SEQ ID NO: 8, SEQ ID NO: 9 or SEQ ID NO: 10. In one aspect, the PDG polypeptide further includes a targeting sequence, such as a nuclear or mitochondrial targeting sequence. In another aspect, the PDG polypeptide further includes a PTD. In yet another aspect, the PDG polypeptide further includes both a targeting sequence and a PTD.

In one embodiment, the PTD includes a HIV TAT peptide. However, the PDG polypeptides are not limited by the type of PTD. A number of PTDs are well known in the art and are described herein. PTDs that can be used with the PDG polypeptides include both naturally occurring PTDs and synthetic (also referred to as “artificial”) peptides. In one embodiment, the PTD is fused to the PDG polypeptide at the carboxy terminus.

The PDG polypeptides also are not limited by the type of NLS or MLS as long as the targeting sequence is capable of delivering a catalytically active enzyme to the appropriate organelle. Numerous organelle targeting sequences are well known in the art and described herein.

When fused to a PDG polypeptide, a targeting sequence can be at the amino or carboxy terminus of the polypeptide as long as the PDG polypeptide retains catalytic activity when delivered to the desired organelle. In some cases, the PDG polypeptide will not be active until the targeting sequence is cleaved and/or delivered to the appropriate organelle. In one embodiment, the targeting sequence is a MLS fused to the amino terminus of the PDG polypeptide. In another embodiment, the targeting sequence is a NLS fused to the carboxy terminus of the PDG polypeptide.

The PDG polypeptides described herein can further include additional domains, such as amino acid sequences useful for facilitating purification and isolation of the PDG polypeptides. In one embodiment, the amino acid sequence is a 6-His tag.

Also provided herein are pharmaceutical compositions including the PDG polypeptides described herein. In one embodiment, the pharmaceutical compositions include a therapeutically effective amount of the PDG polypeptide in a pharmaceutically acceptable carrier.

Further provided herein are isolated polynucleotides encoding the PDG polypeptides described herein. Also provided are vectors including such polynucleotides and cells comprising such polynucleotides. The vectors including PDG polynucleotides can further include other elements, such sequences encoding a selectable marker or protein that facilitates purification of the expressed polypeptide.

Also provided herein is a method for increasing the repair rate of damaged bases in a cell, including contacting a cell in need of DNA repair with a therapeutically effective concentration of an agent comprising an isolated PDG polypeptide described herein, thereby increasing the repair rate of damaged DNA in the cell compared to an untreated cell. In one embodiment, the cell is a cancer cell. In one example, the cancer cells is a keratinocyte carcinoma, such as a basal cell carcinoma or a squamous cell carcinoma. In some embodiments, the cells is a skin cell, such as a keratinocyte, squamous cell or basal cell.

Further provided is a method for increasing the UV-resistance of a cell, comprising contacting the cell with an effective concentration of an agent including an isolated PDG polypeptide described herein, thereby increasing the UV-resistance of the cell compared to an untreated cell.

Further provided is a method of treating a skin disorder in a subject, including contacting the skin of the subject in need treatment with a therapeutically effective concentration of an agent comprising an isolated PDG polypeptide provided herein. In one embodiment, skin disorder is skin cancer. In another embodiment, the skin disorder is psoriasis. In yet another embodiment, the skin disorder is actinic keratosis.

Also provided is a method of treating UV-induced immunosuppression in a subject, including contacting the skin of the subject in need treatment with a therapeutically effective concentration of an agent including an isolated PDG polypeptide described herein.

### IV. Polypeptides and Polynucleotides with PDG Activity

Provided herein are compositions and methods for activation of the BER pathway in mammalian cells for the removal of CPDs by introducing dimer-specific DNA glycosylases. Previous studies involving activation of the BER pathway in human cells involved microinjecting T4-PDG into cells from xeroderma pigmentosum (XP) patients, in which it was observed that following UV irradiation, unscheduled DNA repair synthesis was greatly enhanced. Subsequent strategies were based on expressing the gene encoding T4-PDG in a variety of rodent and human cells that were either repair-deficient or proficient (Francis et al., Mutat. Res. 385(1):59-74, 1997; Kibitel et al., Photochem. Photobiol. 54(5):753-60, 1991; Kusewitt et al., Photochem. Photobiol. 58(3):450-4, 1993; Kusewitt et al., J. Invest. Dermatol. 102(4):485-9, 1994; Kusewitt et al., Mutat. Res. 255(1):1-9, 1991). Collectively, these studies concluded that rates of dimer repair can be greatly accelerated by the expression of T4-Pdg; UV-induced mutagenesis of plasmid can be suppressed; survival of repair-deficient cells containing T4-PDG was increased; and survival of normal repair-proficient cells expressing WT-T4-PDG was always significantly less than the control. The latter data are qualitatively similar to studies reported by Yang et al. (DNA Repair 3(10):1323-34, 2004), which demonstrated that overexpression of either human NTH1 or OGG1 in human lymphoblastoid cells resulted in significantly decreased survival following ionizing radiation exposure. This study demonstrated that decreased survival was due to glycosylase/AP lyases creating double-stranded breaks at sites of lesions in closely opposed DNA strands. Thus, the ability to form cytotoxic double-strand breaks outweighs the positive features of enhanced BER in wild-type cells.

Extrapolation of the decreased survival data in wild-type cells to normal human populations raises concerns about the efficiency and safety of using an enzyme that efficiently promotes double-stranded breaks in UV-irradiated cells. Thus, the present disclosure provides methods of enhancing DNA repair capacity without promoting frequent double-stranded breaks.

The present disclosure provides polypeptides that have PDG activity and polynucleotides encoding such polypeptides. In some embodiments, the polypeptides further include a targeting sequence and a protein transduction domain. Also provided are mutant PDG polypeptides, including mutant T4-PDG and mutant CV-PDG. The mutant PDG polypeptides described herein retain at least partial catalytic activity, but incise CPDs randomly rather than in clusters by destabilizing the pre-catalytic steps of DNA bending and nucleotide flipping.

Polypeptides with PDG Activity

A polypeptide that recognizes the presence of two consecutive damaged bases in a polynucleotide and catalyzes the breakage of the glycosyl bond between the 5′ base and the DNA sugar-phosphate backbone has “glycosylase activity.” Whether a polypeptide has pyrimidine glycosylase activity can be determined by measuring the ability of the polypeptide to cleave the glycosyl bond of the 5′ pyrimidine of a cyclobutane pyrimidine dimer in DNA. Such methods are known to the art. Examples of polypeptides with PDG activity include T4-PDG polypeptides, CV-PDG polypeptides and mutants thereof. T4-PDG and CV-PDG polypeptide sequences include, but are not limited to those provided below and described herein.

Wild-type T4-PDG
(SEQ ID NO: 1)
MTRINLTLVSELADQHLMAEYRELPRVFGAVRKHVANGKRVRDFKISP
TFILGAGHVTFFYDKLEFLRKRQIELIAECLKRGFNIKDTTVQDISDI
PQEFRGDYIPHEASIAISQARLDEKIAQRPTWYKYYGKAIYA
R3Q T4-PDG
(SEQ ID NO: 2)
MTQ3INLTLVSELADQHLMAEYRELPRVFGAVRKHVANGKRVRDFKIS
PTFILGAGHVTFFYDKLEFLRKRQIELIAECLKRGFNIKDTTVQDISD
IPQEFRGDYIPHEASIAISQARLDEKIAQRPTWYKYYGKAIYA
R22Q T4-PDG
(SEQ ID NO: 3)
MTRINLTLVSELADQHLMAEYQ22ELPRVFGAVRKHVANGKRVRDFKI
SPTFILGAGHVTFFYDKLEFLRKRQIELIAECLKRGFNIKDTTVQDIS
DIPQEFRGDYIPHEASIAISQARLDEKIAQRPTWYKYYGKAIYA
R26Q T4-PDG
(SEQ ID NO: 4)
MTRINLTLVSELADQHLMAEYRELPQ26VFGAVRKHVANGKRVRDFKI
SPTFILGAGHVTFFYDKLEFLRKRQIELIAECLKRGFNIKDTTVQDIS
DIPQEFRGDYIPHEASIAISQARLDEKIAQRPTWYKYYGKAIYA
R117Q T4-PDG
(SEQ ID NO: 5)
MTRINLTLVSELADQHLMAEYRELPRVFGAVRKHVANGKRVRDFKISP
TFILGAGHVTFFYDKLEFLRKRQIELIAECLKRGFNIKDTTVQDISDI
PQEFRGDYIPHEASIAISQAQ117LDEKIAQRPTWYKYYGKAIYA
Wild-type CV-PDG
(SEQ ID NO: 6)
MTRVNLVPVQELADQHLMAEFRELKMIPKALARSLRTQSSEKILKKIP
SKFTLNTGHVLFFYDKGKYLQQRYDEIVVELVDRGYKINVDAKLDPDN
VMTGEWYNDYTPTEDAFNIIRARIAEKIAMKPSFYRFTKAKTSNN
R3Q CV-PDG
(SEQ ID NO: 7)
MTQ3VNLVPVQELADQHLMAEFRELKMIPKALARSLRTQSSEKILKKI
PSKFTLNTGHVLFFYDKGKYLQQRYDEIVVELVDRGYKINVDAKLDPD
NVMTGEWYNDYTPTEDAFNIIRARIAEKIAMKPSFYRFTKAKTSNN
R22Q CV-PDG
(SEQ ID NO: 8)
MTRVNLVPVQELADQHLMAEFQ22ELKMIPKALARSLRTQSSEKILKK
IPSKFTLNTGHVLFFYDKGKYLQQRYDEIVVELVDRGYKINVDAKLDP
DNVMTGEWYNDYTPTEDAFNIIRARIAEKIAMKPSFYRFTKAKTSNN
R117Q CV-PDG
(SEQ ID NO: 9)
MTRVNLVPVQELADQHLMAEFRELKMIPKALARSLRTQSSEKILKKIP
SKFTLNTGHVLFFYDKGKYLQQRYDEIVVELVDRGYKINVDAKLDPDN
VMTGEWYNDYTPTEDAFNIIQ117ARIAEKIAMKPSFYRFTKAKTSNN
R119Q CV-PDG
(SEQ ID NO: 10)
MTRVNLVPVQELADQHLMAEFRELKMIPKALARSLRTQSSEKILKKIP
SKFTLNTGHVLFFYDKGKYLQQRYDEIVVELVDRGYKINVDAKLDPDN
VMTGEWYNDYTPTEDAFNIIRAQ119IAEKIAMKPSFYRFTKAKTSNN

In some examples, the PDG amino acid sequence includes 20 to 130 amino acids of SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9 or SEQ ID NO: 10, such as 30 consecutive amino acids, 40 consecutive amino acids, 50 consecutive amino acids, 60 consecutive amino acids, 70 consecutive amino acids, 80 consecutive amino acids, 90 consecutive amino acids, 100 consecutive amino acids, 110 consecutive amino acids, 115 consecutive amino acids, 120 consecutive amino acids, 125 consecutive amino acids of any one of these sequences. However, the modified PDGs retain PDG activity.

In one embodiment, a polypeptide of the present disclosure also has apurinic/apyrimidinic lyase activity (AP lyase activity). A polypeptide with AP lyase activity has the ability to catalyze a β-elimination reaction on an abasic site containing DNA, resulting in an α,β unsaturated aldehyde. A polypeptide having pyrimidine glycosylase activity and AP lyase activity is referred to herein as a “pyrimidine glycosylase/AP lyase,” and has “pyrimidine glycosylase/AP lyase activity.” In a particular example, a polypeptide of the present disclosure has pyrimidine glycosylase/AP lyase activity, a targeting sequence, and a protein transduction domain.

Whether a polypeptide has pyrimidine glycosylase/AP lyase activity can be determined by measuring the ability of the polypeptide to incise a target polynucleotide containing damaged bases in the presence of a buffer. The target polynucleotide contains damaged bases, such as UV radiation induced pyrimidine dimers. In an example, the target polynucleotide is present at a concentration of from about 0.1 nM to about 10 nM. The putative glycosylase/AP lyase is present at a concentration of from about 0.01 nM to about 100 nM. Buffers in which a glycosylase/AP lyase is active are suitable for the assay. In a particular example, the buffer includes about 25 mM NaH₂PO₄ and the pH is from about 6.5 to about 7.5, such as about 6.8. In certain examples, the buffer contains from about 10 mM NaCl to about 125 mM NaCl, such as about 100 mM NaCl. In one embodiment, the buffer contains from about 1 mM EDTA to about 10 mM EDTA. In one embodiment, the buffer contains from about 0.01 mg/mL bovine serum albumin (BSA) to about 1 mg/mL BSA. The temperature of the assay is about 37° C. The assay can be carried out for at least about 10 seconds to no greater than about 8 hours. In one embodiment, the assay is about 30 minutes.

A polypeptide having pyrimidine glycosylase/AP lyase activity will cause the mobility of the target polynucleotide to change relative to the polynucleotide that has not been exposed to the polypeptide. The polypeptide may be present in a crude cellular extract, or the polypeptide can be isolated or purified. Since polypeptides identified in this assay as having pyrimidine glycosylase/AP lyase activity function on UV-irradiated DNAs, these polypeptides identify cyclobutane pyrimidine dimers, and are likely to be active on other UV-induced photoproducts including FapyA and FapyG.

Examples of polypeptides having pyrimidine glycosylase activity include amino acid sequences present in the chlorella virus isolate PB CV-1 pyrimidine dimer-specific glycosylase (CV-PDG; SEQ ID NO: 6) and mutants thereof (for example, SEQ ID NOs: 7-10); and the Bacteriophage T4 pyrimidine dimer-specific glycosylase (T4-PDG; SEQ ID NO: 1) and mutants thereof (for example, SEQ ID NOs: 2-5). In some cases, the PDG polypeptides exhibit altered catalytic activity, such as pyrimidine glycosylase activity and/or AP lyase activity.

The present disclosure further includes polypeptides having pyrimidine glycosylase activity, such as pyrimidine glycosylase/AP lyase activity, and amino acid identity with the amino acid sequence of SEQ ID NO: 1 or SEQ ID NO: 6. Amino acid identity is defined in the context of a comparison between a polypeptide, such as SEQ ID NO: 1 or SEQ ID NO: 6, and is determined by aligning the residues of the two amino acid sequences to optimize the number of identical amino acids along the lengths of their sequences; gaps in either or both sequences are permitted in making the alignment in order to optimize the number of identical amino acids, although the amino acids in each sequence must nonetheless remain in their proper order.

### Polynucleotides

Also disclosed herein are polynucleotides that encode polypeptides that have PDG activity. A polynucleotide is a polymeric form of nucleotides of any length, either ribonucleotides or deoxynucleotides, and includes both double- and single-stranded DNA and RNA. A polynucleotide may include nucleotide sequences having different functions, including, for instance, coding sequences, and non-coding sequences such as regulatory sequences. Coding sequence, non-coding sequence, and regulatory sequence are defined below. A polynucleotide can be obtained directly from a natural source, or can be prepared with the aid of recombinant, enzymatic, or chemical techniques. A polynucleotide can be linear or circular in topology. For example, a polynucleotide can be a portion of a vector, such as an expression or cloning vector, or a fragment.

The present disclosure also provides polynucleotides encoding a polypeptide of the present disclosure, such as, for example, a polypeptide having pyrimidine glycosylase activity and/or pyrimidine glycosyalse/AP lyase activity, and a targeting sequence, such as, an exogenous targeting sequence. A polynucleotide can include nucleotide sequences having different functions, including for instance coding sequences, and non-coding sequences such as regulatory sequences. “Coding sequence” and “coding region” are used interchangeably and refer to a polynucleotide that encodes a polypeptide and, when placed under the control of appropriate regulatory sequences expresses the encoded polypeptide. The boundaries of a coding region are generally determined by a translation start codon at its 5′ end and a translation stop codon at its 3′ end. A regulatory sequence is a nucleotide sequence that regulates expression of a coding region to which it is operably linked. Nonlimiting examples of regulatory sequences include promoters, transcription initiation sites, translation start sites, translation stop sites, and terminators. “Operably linked” refers to a juxtaposition wherein the components so described are in a relationship permitting them to function in their intended manner. A regulatory sequence is “operably linked” to a coding region when it is joined in such a way that expression of the coding region is achieved under conditions compatible with the regulatory sequence.

Polynucleotides encoding a polypeptide of the invention can be obtained from a microbe, such as Neisseria mucosa and Bacillus sphearicus, or a microbe harboring a virus that produces a polypeptide having pyrimidine glycosylase activity and/or pyrimidine glycosylase/AP lyase activity. Methods for isolating a polynucleotide encoding a polypeptide employs standard cloning techniques known to the art (see, for example, Sambrook et al., Molecular Cloning: A Laboratory Manual., Cold Spring Harbor Laboratory Press (1989) or Ausubel et al., (Eds.) Current Protocols in Molecular Biology, John Wiley & Sons, Inc. New York, N.Y. (1994)).

Examples of polynucleotides include those encoding the Chlorella virus isolate PBCV-1 pyrimidine dimer-specific glycosylase (CV-PDG, Genbank Accession No. AF128160, SEQ ID NO: 26); and the Bacteriophage T4 pyrimidine dimer-specific glycosylase (T4-PDG, nucleotides 1777-2193 of Genbank Accession No. X04567, SEQ ID NO: 27).

### Targeting Sequence

The polypeptides of the present disclosure also include a targeting sequence.

In one embodiment, the targeting sequence is an exogenous targeting sequence. As used herein, a “targeting sequence” is a polypeptide that is fused to a polypeptide having pyrimidine glycosylase activity, such as pyrimidine glycosylase/AP lyase activity. As used herein, “exogenous targeting sequence” refers to a foreign targeting sequence, i.e., a targeting sequence that is not normally fused to the polypeptide having pyrimidine glycosylase activity, such as pyrimidine glycosylase/AP lyase activity. Targeting sequences cause the polypeptide to which they are fused to migrate from the cytoplasm of a cell to an organelle. In one aspect, the targeting sequence is a NLS that causes migration into the nucleus. During the transit of the polypeptide that includes a NLS to the nucleus of a cell, the NLS may be cleaved. The disclosure is not limited by the type of NLS that is fused to the pyrimidine glycosylase, and many NLSs are known to the art (see, for instance, (Moroianu, J. Cell. Biochem. Suppl. 32/33: 76-83, 1999). An NLS can be present in any location in a polypeptide of the present disclosure provided the presence of the NLS does not inhibit the pyrimidine glycosylase activity of the polypeptide after the pyrimidine glycosylase is delivered to the nucleus. In one embodiment, a NLS is present at the carboxy-terminal end of a pyrimidine glycosylase. In one embodiment, the NLS is a consensus NLS, having the amino acid sequence PKKRKRRL (SEQ ID NO: 11). In another embodiment the NLS is PKKKRKRL (SEQ ID NO: 12). The NLS included in the PDG polypeptides need not have 100% sequence identity to SEQ ID NO: 10 or SEQ ID NO: 11. NLSs having one or more amino acid substitutions relative to SEQ ID NO: 10 or SEQ ID NO: 11 are contemplated. However, the modified NLSs retain the capacity to direct the polypeptide to which they are fused to the nucleus.

In addition, a number of other NLSs are well known in the art, which can be fused to PDG. Representative NLSs include, but are not limited to, Large T (PKKKRKVC; SEQ ID NO: 28); MA-NLS1 (GKKKYKLKH; SEQ ID NO: 29); MA-NLS2 (KSKKKAQ; SEQ ID NO: 30); IN-NLS (KRK; and KELKQKQITK; SEQ ID NO: 31); Vpr N (NEWTLELLEELKNEAVRHF; SEQ ID NO: 32); Vpr C (RHSRIGVTRGRRARNGASRS; SEQ ID NO: 33); Rev NLS (RQARRNRRRRWR; SEQ ID NO: 34). H2B (GKKRSKV; SEQ ID NO: 35); v-Jun (KSRKRKL; SEQ ID NO: 36); nucleoplasmin (RPAATKKAGQAKKKKLDK; SEQ ID NO: 37); NIN2 (RKKRKTEEESPLKDKAKKSK; SEQ ID NO: 38); or SWI5 (KKYENVVIKRSPRKRGRPRK; SEQ ID NO: 39) (see, for example, U.S. Pre-Grant Publication No. 2005/0220796, herein incorporated by reference). It will be appreciated that the NLS can also be selected from those listed in NLSdb, a database of NLSs, which is available online

In another aspect, the targeting sequence is a MLS that causes migration into mitochondria. The disclosure is not limited by the type of MLS that is fused to the pyrimidine glycosylase. Typically, a MLS is fused to the amino-terminal end of a polypeptide described herein. In those aspects of the disclosure where an MLS is fused to the amino terminal end of a pyrimidine glycosylase, the MLS is cleaved during the transit of the polypeptide that includes the MLS into a cell's mitochondria. In some aspects, the pyrimidine glycosylase, such as pyrimidine glycosylase/AP lyase, of the present disclosure are inactive while the MLS is fused, but are active after the MLS is cleaved upon transit into a mitochondrion. In some cases, the MLS comprises one or more modifications to allow for proper cleavage from the fusion protein. Examples of MLSs that can be used include those present in polypeptides that are targeted to the mitochondria, including, for instance, mitochondrial tryphtophanyl-tRNA synthetases (Jorgensen et al., J. Biol. Chem. 275:16820-16826, 2000), mitochondrial uracil DNA glycosylase (Otterlei et al., Nucleic Acids Res. 26:4611-4617, 1998), manganese superoxide dismutase (Wispe et al., Biochim. Biophys. Acta 994:30-36, 1989), and ornithine transcarbamylase (Horwich et al., Science 224:1068-1074, 1984), among others. Examples of MLSs that can be used in the present disclosure include MALHSMRKARERWSFIRA (SEQ ID NO: 13) and MGVFCLGFWGLGRKLRTFGKGPKQLLSRLCGDHLQ (SEQ ID NO: 14). The MLS fused to the PDG polypeptide need not share 100% sequence identity with SEQ ID NO: 13 or SEQ ID NO: 14. MLSs having one or more amino acid substitutions relative to SEQ ID NO: 13 or SEQ ID NO: 14 also are contemplated herein, as long as the MLS retains the capacity to direct a polypeptide sequence to the mitochondria.

Other exemplary organelle localization sequences include those described in Emanuelson et al. (J. of Mol. Biol. 300(4):1005-1016, 2000) and in Cline and Henry (Annu. Rev. Cell Dev. Biol. 12:1-26, 1996), each of which is herein incorporated by reference. It will be appreciated that the entire sequence need not be included, and modifications including truncations of these sequences are within the scope of the disclosure provided the sequences operate to direct a linked molecule to a specific organelle, cell, or tissue.

Whether a polypeptide described herein is delivered to the appropriate organelle can be determined by several methods. The polypeptide can be introduced to a eukaryotic cell by, for instance, by microinjection of the polypeptide into the cytoplasm of the cell. Alternatively, the polypeptide is introduced to the cytoplasm of the cell as a composition including the polypeptide and a pharmaceutically acceptable carrier, such as a liposome, phospholipid, or pH-activated lipid. Pharmaceutically acceptable carriers are described herein. To determine whether the introduced polypeptide is targeted to the nucleus or the mitochondria of a cell, the appropriate organelle can be isolated, and the amount of the polypeptide in the organelle determined. Alternatively, immunofluorescence analysis with an antibody that binds to the polypeptide can be used to determine the intracellular distribution of the polypeptide after it is introduced.

When determining whether a polypeptide of the disclosure is delivered to the appropriate organelle, the polypeptide may be introduced to the cell as a polynucleotide encoding the polypeptide. The polypeptide is expressed from the polynucleotide and translated in the cytoplasm of the cell. The targeting of the polypeptide to the nucleus or mitochondria of a cell can be determined as described above. It should be noted that as used herein, a polynucleotide encoding the polypeptide is used ex vivo to test whether a polypeptide is delivered to the nucleus or a mitochondrion; polynucleotides are not used for the in vivo delivery of polypeptides of the present disclosure. Polynucleotides are described herein.

Whether the polypeptide of the present disclosure retains pyrimidine glycosylase activity, such as pyrimidine glycosylase/AP lyase activity, once transported into the organelle can be determined by several methods. The polypeptide can be introduced to the cell as described herein, including introduction as a polypeptide and introduction as a polynucleotide that encodes the polypeptide. To measure activity after introduction to the cell, the appropriate organelle can be isolated, the polypeptide isolated from the organelle, and the activity of the isolated polypeptide determined. Alternatively, the repair rate of damaged DNA in the cell can be determined using, for instance, coding sequence-specific repair assays, photoproduct removal, and/or quantitative PCR.

### Protein Transduction Domain (PTD)

PTDs constitute a family of polypeptides that facilitate protein transduction across membranes in a receptor-independent manner (Wadia and Dowdy, Curr. Protein Pept. Sci. 4(2):97-104, 2003). This phenomena was originally described for the human immunodeficiency virus (HIV)-encoded transactivator of transcription (TAT) protein, which was shown to cross membranes and initiate transcription. It was then discovered that the portion of the TAT protein that was required for the transduction of the protein was only an 11 amino acid polypeptide: tyrosine (Y), glycine (G), arginine (R), lysine (K), lysine (K), arginine (R), arginine (R), glutamine (Q), arginine (R), arginine (R), arginine (R) (YGRKKRRQRRR; SEQ ID NO: 40), hereinafter referred to as the TAT peptide. When fused with other proteins, the TAT peptide has been demonstrated to deliver these proteins, varying in size from 15 to 120 kDa, into cells in tissue culture (Frankel and Pabo, Cell 55(6):1189-93, 1988; Green and Loewenstein, J. Gen. Microbiol. 134(3):849-55, 1988; Vives et al., J. Biol. Chem. 272(25):16010-7, 1997; Yoon et al., J. Microbiol. 42(4):328-35, 2004; Cai et al., Eur. J. Pharm. Sci. 27(4):311-9, 2006).

Other TAT polypeptide sequences include, but are not limited to RKKRRQRRR (SEQ ID NO: 41); KKKKKKKKK (SEQ ID NO: 42); or RRRRRRRRR (SEQ ID NO: 43). The PDG polypeptides provided herein need not comprise a TAT peptide sequence having 100% identity to SEQ ID NO: 40, 41, 42 or 43. The current disclosure contemplates use of a modified or variant TAT peptide in which one or more amino acids differ from one of the TAT peptide sequences provided herein. However, the modified or variant TAT peptide contemplated for use retains the capacity to facilitate protein transduction across membranes. Methods of preparing TAT fusion proteins and expression vectors comprising TAT fusion proteins are well known in the art (see, for example, U.S. Pat. Nos. 7,094,407 and 7,060,673, herein incorporated by reference in their entirety).

Thus, as described herein, TAT polypeptides are useful for delivery of biologically active enzymes in organisms. Provided herein are TAT polypeptides fused to PDG glycosylases that are targeted to either the nucleus or mitochondria (using an NLS or MTS). When these fusion proteins are applied to the outer portion of the skin (stratum corneum), the PDG polypeptides will penetrate through this layer of dead cells and distribute to all cell types in the epidermal and dermal layers. Once inside the cells, the PDG will localize to the appropriate intracellular organelle and initiate repair of CPDs and other oxidative DNA lesions. This will serve to both minimize mutations that can lead to cancer and prevent UV-induced immunosuppression, thus allowing the natural immune system to kill emerging cancer cells.

Other PTDs are known in the art and can be used in the compositions and methods described herein. Examples of such PTDs include, but are not limited to, peptides from theVP22 protein of herpes simplex virus (HSV) type 1 (Elliott et al., Cell 88:223-233, 1997); the UL-56 protein of HSV-2 (U.S. Pre-Grant Publication No. 2006/0099677); the Vpr protein of HIV-1 (U.S. Pre-Grant Publication No. 2005/0287648); the third helix of the Drosophila Antennapedia homebox gene (Derossi et al., J. Biol. Chem. 269:10444-10450, 1994; Schwarze et al., Trends Pharmacol. Sci. 21:45-48, 2000); the transportan protein (Pooga, FASEB J. 12:67-77, 1998; Hawiger, Curr. Opin. Chem. Biol. 3:89-94, 1999). A number of artifical peptides also are know to function as PTDs, such as poly-arginine, poly-lysine and others (see, for example, U.S. Pre-Grant Publication Nos. 2006/0106197; 2006/0024331; 2005/0287648; and 2003/0125242; Zhibao et al., Mol. Ther. 2:339-347, 2000; and Laus et al. Nature Biotechnol. 18:1269-1272, 2000). Each of the above-listed publications, and PTD sequences disclosed therein, is herein incorporated by references in its entitirety.

The use of a PTD in the PDG fusion protein eliminates the need to package proteins and enzymes into liposomes for delivery into skin. Potential commercial uses for PDG fusion proteins delivered in such a manner include sunscreens, delivery of antiaging protein, and treatment of skin disease.

### Additional Domains

Optionally, a polypeptide of the present disclosure further includes a series of consecutive amino acids encoding a domain that facilitates the isolation and purification of the polypeptide. An “isolated” polypeptide or polynucleotide means a polypeptide or polynucleotide that has been either removed from its natural environment, produced using recombinant techniques, or chemically or enzymatically synthesized. In one embodiment, a polypeptide or polynucleotide of this disclosure is purified, i.e., essentially free from any other polypeptide or polynucleotide and associated cellular products or other impurities. For instance, domains that are useful in the isolation of a polypeptide that has glycosylase activity, such as glycosylase/AP lyase activity, include a histidine domain (which can be isolated using nickel-chelating resins), an S-peptide domain (which can be isolated using an S-protein, see Kim, J.-S. et al. Protein Sci. 2:348-356, 1993), and a chitin binding domain (which can bind to chitin beads, see Chong et al. Gene 192:271-281, 1997; and Watanabe et al. J. Bacteriol. 176:4465-4472, 1994). In one embodiment, the domain is present at the carboxy terminal end of the polypeptide. In one embodiment, the domain can be cleaved from the remainder of the polypeptide (e.g., the polypeptide having pyrimidine glycosylase activity, such as pyrimidine glycosylase/AP lyase activity, fused to a targeting sequence, such as an exogenous targeting sequence) by the use of a protease or self-cleaving sequence.

### V. Expression Vectors

A polynucleotide encoding a PDG polypeptide can be included in an expression vector to direct expression of the PDG nucleic acid sequence. The term vector includes, but is not limited to, plasmid vectors, viral vectors, cosmid vectors, or artificial chromosome vectors. Typically, a vector is capable of replication in a bacterial host, for instance E. coli, or in a eukaryotic cell. In one embodiment, the vector is a plasmid vector. Selection of a vector depends upon a variety of desired characteristics in the resulting construct, such as a selection marker, vector replication rate, and the like. Suitable host cells for cloning or expressing the vectors herein are prokaryotic or eukaryotic cells.

Other expression control sequences including appropriate promoters, enhancers, transcription terminators, a start codon (i.e., ATG) in front of a protein-encoding gene, splicing signal for introns, maintenance of the correct reading frame of that gene to permit proper translation of mRNA, and stop codons can be included with a polynucleotide sequence in an expression vector. Generally expression control sequences include a promoter, a minimal sequence sufficient to direct transcription.

The expression vector typically contains an origin of replication and a promoter. In some instances, the expression vector comprises specific genes which allow phenotypic selection of the transformed cells (such as an antibiotic resistance cassette). Generally, the expression vector will include a promoter. The promoter can be inducible or constitutive. The promoter can be tissue specific. In one embodiment, the promoter is a heterologous promoter. In one example, the polynucleotide encoding the PDG polypeptide is located downstream of the desired promoter. Optionally, an enhancer element is also included, and can generally be located anywhere on the vector and still have an enhancing effect. However, the amount of increased activity will generally diminish with distance.

An expression vector can optionally include a ribosome binding site (a Shine Dalgarno site for prokaryotic systems or a Kozak site for eukaryotic systems) and a start site to initiate translation of the transcribed message to produce the polypeptide. It can also include a termination sequence to end translation. A termination sequence is typically a codon for which there exists no corresponding aminoacetyl-tRNA, thus ending polypeptide synthesis. The polynucleotide used to transform the host cell can optionally further include a transcription termination sequence. The rrnB terminators, T1 and T2, are an often used terminator that is incorporated into bacterial expression systems. Transcription termination sequences in vectors for eukaryotic cells typically include a polyadenylation signal 3′ of the coding region.

Also useful are expression vectors that provide for transient expression in eukaryotic cells of a coding sequence encoding a polypeptide disclosed herein. In general, transient expression involves the use of an expression vector that is able to replicate efficiently in a host cell, such that the host cell accumulates many copies of the expression vector and, in turn, synthesizes high levels of a desired polypeptide encoded by the expression vector. Transient expression systems, including a suitable expression vector and a host cell, allow for the convenient positive identification of polypeptides that are targeted to the appropriate organelle. Methods for the transient expression of coding regions are well known in the art.

Construction of vectors containing a polynucleotide of the disclosure employs standard ligation techniques well known in the art (see, for example, Sambrook et al., Molecular Cloning: A Laboratory Manual., Cold Spring Harbor Laboratory Press (1989) or Ausubel et al., (Eds.) Current Protocols in Molecular Biology, John Wiley & Sons, Inc. New York, N.Y. (1994). Vectors suitable for use include, but are not limited to, pTYB2 (New England Biolabs; Garvish and Lloyd, J. Mol. Biol. 295:479-7488, 2000), pcDNA3.1 (Invitrogen, Carlsbad, Calif.) and pET-22b (Novagen).

Expression vectors including a polynucleotide encoding a PDG polypeptide can be used to transform host cells. Hosts can include isolated microbial, yeast, insect and mammalian cells, as well as cells located in the organism, such as a human. Biologically functional viral and plasmid DNA vectors capable of expression and replication in a host are known in the art, and can be used to transfect any cell of interest.

Vectors suitable for expression of DNA repair enzymes and fusion proteins thereof are known in the art (see, for example, U.S. Pre-Grant Publication No. 2005/0220796 and U.S. Pat. No. 6,723,548, each of which is herein incorporated by reference in its entirety).

### VI. Pharmaceutical Compositions

The PDG polypeptides disclosed herein may be included in pharmaceutical compositions (including therapeutic and prophylactic formulation), typically combined together with one or more pharmaceutically acceptable vehicles or carriers, and optionally, other therapeutic ingredients. Typically, the composition includes a pharmaceutically acceptable carrier when the composition is used as described below in “Methods of Use.” The PDG polypeptides disclosed herein may be combined and/or used in combination with other therapeutic agents, different from the subject PDG polypeptides depending on the specific condition or disease being treated.

Pharmaceutical compositions including a disclosed PDG polypeptide can be administered to subjects by a variety of modes, including topical administration, parental administration (for instance intramuscular, intraperitoneal, or intravenous), oral, transdermal, nasal, or aerosol. The formulations may be conveniently presented in unit dosage form and may be prepared by methods well known in the art of pharmacy. Methods of preparing a pharmaceutical composition include associating the active compound (e.g., a disclosed PDG polypeptide) into association with a carrier that constitutes one or more accessory ingredients. In general, the formulations are prepared by uniformly and intimately bringing the active compound into association with a liquid carrier, a finely divided solid carrier, or both, and then, if necessary, shaping the product into the desired formulations.

The compositions can be administered as needed, such as at least once per day. The amount of active ingredient that may be combined with the carrier materials to produce a single dosage form will vary depending upon the subject treated and the particular mode of administration. A typical preparation will contain from about 5% to about 95% active compound (w/w). In one example, such preparations contain from about 20% to about 80% active compound. The amount of active compound in such therapeutically useful compositions is such that the dosage level will be effective to prevent or suppress the condition the subject has or is at risk for. Such conditions are described hereinbelow.

Formulations suitable for topical administration can include dusting powders, ointments, cremes, gels or sprays for the administration of the active compound to cells, such as skin cells. Such formulations may optionally include an inorganic pigment, organic pigment, inorganic powder, organic powder, hydrocarbon, silicone, ester, triglyceride, lanolin, wax, cere, animal or vegetable oil, surfactant, polyhydric alcohol, sugar, vitamin, amino acid, antioxidant, free radical scavenger, ultraviolet light blocker, sunscreen agents, preservative, fragrance, thickener, or combinations thereof.

As one example, the active compounds of the present disclosure can be used in cosmetic formulations (e.g., skincare cream, sunscreen, decorative make-up products, and other dermatological compositions) in various pharmaceutical dosage forms, and especially in the form of oil-in-water or water-in-oil emulsions, solutions, gels, or vesicular dispersions. The cosmetic formulations may take the form of a cream which can be applied either to the face or to the scalp and hair, as well as to the human body, in particular those portions of the body that are chronically exposed to sun. They can also serve as a base for a lipstick.

In some cosmetic formulations, additives can be included such as, for example, preservatives, bactericides, perfumes, antifoams, dyes, pigments which have a coloring action, surfactants, thickeners, suspending agents, fillers, moisturizers, humectants, fats, oils, waxes or other customary constituents of a cosmetic formulation, such as alcohols, polyols, polymers, foam stabilizers, electrolytes, organic solvents, or silicone derivatives.

Cosmetic formulations typically include a lipid phase and often an aqueous phase. The lipid phase can be chosen from the following group of substances: mineral oils, mineral waxes, such as triglycerides of capric or of caprylic acid, castor oil; fats, waxes and other natural and synthetic fatty substances, esters of fatty acids with alcohols of low C number, for example with isopropanol, propylene glycol or glycerol, or esters of fatty alcohols with alkanoic acids of low C number or with fatty acids; alkyl benzoates; silicone oils, such as dimethylpolysiloxanes, diethylpolysiloxanes, diphenylpolysiloxanes and mixed forms thereof.

If appropriate, the aqueous phase of the formulations according to the present disclosure include alcohols, diols or polyols of low C number and ethers thereof, such as ethanol, isopropanol, propylene glycol, glycerol, ethylene glycol, ethylene glycol monoethyl or monobutyl ether, propylene glycol monomethyl, monoethyl or monobutyl ether, diethylene glycol monomethyl or monoethyl ether and analogous products, furthermore alcohols of low C number, for example ethanol, isopropanol, 1,2-propanediol and glycerol, and, in particular, one or more thickeners, such as silicon dioxide, aluminium silicates, polysaccharides and derivatives thereof, for example hyaluronic acid, xanthan gum and hydroxypropylmethylcellulose, or poly-acrylates.

An exemplary cosmetic formulation is a sunscreen composition. A sunscreen can additionally include at least one further UVA filter and/or at least one further UVB filter and/or at least one inorganic pigment, such as an inorganic micropigment. The UVB filters can be oil-soluble or water-soluble. Oil-soluble UVB filter substances can include, for example: 3-benzylidenecamphor derivatives, such as 3-(4-methylbenzylidene)camphor and 3-benzylidenecamphor; 4-aminobenzoic acid derivatives, such as 2-ethylhexyl 4-(dimethylamino)benzoate and amyl 4-(dimethylamino)benzoate; esters of cinnamic acid, such as 2-ethylhexyl 4-methoxycinnamate and isopentyl 4-methoxycinnamate; derivatives of benzophenone, such as 2-hydroxy-4-methoxybenzophenone, 2-hydroxy-4-methoxy-4′-methylbenzophenone and 2,2′-dihydroxy-4-methoxybenzophenone; esters of benzalmalonic acid, such as di(2-ethylhexyl)-4-methoxybenzalmalonate. Water-soluble UVB filter substances can include the following: salts of 2-phenylbenzimidazole-5-sulphonic acid, such as its sodium, potassium or its triethanolammonium salt, and the sulphonic acid itself; sulphonic acid derivatives of benzophenones, such as 2-hydroxy-4-methoxybenzophenone-5-sulphonic acid and salts thereof; sulphonic acid derivatives of 3-benzylidenecamphor, such as, for example, 4-(2-oxo-3-bornylidenemethyl)benzenesulphonic acid, 2-methyl-5-(2-oxo-3-bornylidenemethyl)benzenesulphonic acid and salts thereof. The list of further UVB filters mentioned which can be used in combination with the active agent(s) according to the disclosure is not intended to be limiting.

Formulations for parenteral administration include a sterile aqueous preparation of the composition, or dispersions of sterile powders that include the composition, which in an example are isotonic with the blood of the recipient. Isotonic agents that can be included in the liquid preparation include sugars, buffers, and sodium chloride. Solutions of the composition can be prepared in water, and optionally mixed with a nontoxic surfactant. Dispersions of the composition can be prepared in water, ethanol, a polyol (such as glycerol, propylene glycol, liquid polyethylene glycols, and the like), vegetable oils, glycerol esters, and mixtures thereof. The final dosage form can be sterile, fluid and stable under the conditions of manufacture and storage. The desired fluidity can be achieved, for example, by using liposomes, by employing the appropriate particle size in the case of dispersions, or by using surfactants. Sterilization of a liquid preparation can be achieved by any convenient method that preserves the bioactivity of the composition, such as by filter sterilization. Methods for preparing powders include vacuum drying and freeze drying of the sterile injectable solutions. Subsequent microbial contamination can be prevented using various antimicrobial agents, for example, antibacterial, antiviral and antifungal agents including parabens, chlorobutanol, phenol, sorbic acid, thimerosal, and the like. Absorption of the composition by the animal over a prolonged period can be achieved by including agents for delaying, for example, aluminum monostearate and gelatin.

Formulations of the present disclosure suitable for oral administration may be presented as discrete units such as tablets, troches, capsules, lozenges, wafers, or cachets, each containing a predetermined amount of the active compound as a powder or granules, as liposomes containing the active compound, or as a solution or suspension in an aqueous liquor or non-aqueous liquid such as a syrup, an elixir, an emulsion or a draught.

The tablets, troches, pills, capsules, and the like can also contain one or more of the following: a binder such as gum tragacanth, acacia, corn starch or gelatin; an excipient such as dicalcium phosphate; a disintegrating agent such as corn starch, potato starch, alginic acid and the like; a lubricant such as magnesium stearate; a sweetening agent such as sucrose, fructose, lactose or aspartame; and a natural or artificial flavoring agent. When the unit dosage form is a capsule, it can further contain a liquid carrier, such as a vegetable oil or a polyethylene glycol. Various other materials can be present as coatings or to otherwise modify the physical form of the solid unit dosage form. For instance, tablets, pills, or capsules can be coated with gelatin, wax, shellac, or sugar and the like. A syrup or elixir can contain one or more of a sweetening agent, a preservative such as methyl- or propylparaben, an agent to retard crystallization of the sugar, an agent to increase the solubility of any other ingredient, such as a polyhydric alcohol, for example glycerol or sorbitol, a dye, and flavoring agent. The material used in preparing any unit dosage form is substantially nontoxic in the amounts employed. The active compound can be incorporated into sustained-release preparations and devices.

In accordance with the various treatment methods of the disclosure, the compound can be delivered to a subject in a manner consistent with conventional methodologies associated with management of the disorder for which treatment or prevention is sought. In accordance with the disclosure herein, a prophylactically or therapeutically effective amount of the compound and/or other biologically active agent is administered to a subject in need of such treatment for a time and under conditions sufficient to prevent, inhibit, and/or ameliorate a selected disease or condition or one or more symptom(s) thereof.

Typical subjects intended for treatment with the PDG polypeptides and methods of the present disclosure include humans, as well as non-human primates and other animals. To identify subjects for prophylaxis or treatment according to the methods of the disclosure, accepted screening methods are employed to determine risk factors associated with a targeted or suspected disease of condition (for example, skin cancer) or to determine the status of an existing disease or condition in a subject. These screening methods include, for example, diagnostic methods, such as various ELISA and other immunoassay methods, which are available and well known in the art to detect and/or characterize disease-associated markers. These and other routine methods allow the clinician to select patients in need of therapy using the methods and pharmaceutical compositions of the disclosure.

The administration of a PDG polypeptide of the disclosure can be for either prophylactic or therapeutic purpose. When provided prophylactically, the PDG polypeptide is provided in advance of any symptom. The prophylactic administration of the compound serves to prevent or ameliorate any subsequent disease process. When provided therapeutically, the compound is provided at (or shortly after) the onset of a symptom of disease or infection.

For prophylactic and therapeutic purposes, the PDG polypeptide can be administered to the subject such as by topical delivery over an extended time period, or in a repeated administration protocol (for example, by an hourly, daily or weekly, repeated administration protocol). The therapeutically effective dosage of the compound can be provided as repeated doses within a prolonged prophylaxis or treatment regimen that will yield clinically significant results to alleviate one or more symptoms or detectable conditions associated with a targeted disease or condition as set forth herein. Determination of effective dosages in this context is typically based on animal model studies followed up by human clinical trials and is guided by administration protocols that significantly reduce the occurrence or severity of targeted disease symptoms or conditions in the subject. Suitable models in this regard include, for example, murine, rat, porcine, feline, non-human primate, and other accepted animal model subjects known in the art. Alternatively, effective dosages can be determined using in vitro models (for example, immunologic and histopathologic assays). Using such models, only ordinary calculations and adjustments are required to determine an appropriate concentration and dose to administer a therapeutically effective amount of a PDG polypeptide (for example, amounts that are effective to alleviate one or more symptoms of a targeted disease or condition or to prevent UV-induced DNA damage). In alternative embodiments, an effective amount or effective dose of a PDG polypeptide may simply inhibit or enhance one or more selected biological activities correlated with a disease or condition.

The actual dosage of a PDG polypeptide will vary according to factors such as the disease indication and particular status of the subject (for example, the subject's age, size, fitness, extent of symptoms, susceptibility factors, and the like), time and route of administration, other drugs or treatments being administered concurrently. Dosage regimens can be adjusted to provide an optimum prophylactic or therapeutic response. A therapeutically effective amount is also one in which any toxic or detrimental side effects of the compound and/or other biologically active agent is outweighed in clinical terms by therapeutically beneficial effects. Dosages of the pharmaceutical compositions of the presented disclosure are typically from about 0.01 mg/kg up to about 0.10 mg/kg. Dosage can be varied by the attending clinician to maintain a desired concentration at a target site. Higher or lower concentrations can be selected based on the mode of delivery, for example, topical, trans-epidermal, rectal, oral, pulmonary, intranasal delivery, intravenous or subcutaneous delivery. Dosage can also be adjusted based on the release rate of the administered formulation, for example, sustained release oral versus injected particulate or transdermal delivery formulations, and so forth. To achieve the same serum concentration level, for example, slow-release particles with a release rate of 5 nM (under standard conditions) would be administered at about twice the dosage of particles with a release rate of 10 nM.

### VII. Kits

Also disclosed are kits, packages, and multi-container units containing the herein described pharmaceutical compositions, such as pharmaceutical compositions containing one or more of the PDG polypeptides, active ingredients, and/or means for administering the same for use in the prevention and treatment of diseases and other conditions in mammalian subjects. In one embodiment, these kits include a container or formulation that contains one or more of the PDG polypeptides described herein. In one example, this component is formulated in a pharmaceutical preparation for topical delivery to a subject. The PDG polypeptide is optionally contained in a bulk dispensing container or unit or multi-unit dosage form. Packaging materials optionally include a label or instruction indicating for what treatment purposes and/or in what manner the pharmaceutical agent packaged therewith can be used.

### VIII. Methods of Use

The present disclosure is further directed to methods for repairing damaged bases in cells, such as skin cells; methods of enhancing UV-resistance in wild-type cells; methods of treating skin cancer; methods of treating skin disorders such as psoriasis or actinic keratosis; and methods of treating UV-induced immunosuppression. The methods described herein comprise delivery of a PDG polypeptide to a cell. In one embodiment, the cell is a eukaryotic cell, such as a human cell. Cell types that are useful in the methods disclosed herein include cells present in the epidermis, including, for instance, keratinocytes, squamous cells, basal cells, melanocytes, and Langerhans' cells.

Treatment of the conditions (such as skin cancer) described herein can be prophylactic or, alternatively, can be initiated after the development of a condition described herein. Treatment that is prophylactic, for instance, can be initiated before a subject manifests symptoms of a condition and/or before exposure to an agent that damages DNA, such as UV light, oxidative stress, alkylation damage and deamination. Treatment prior to the development of the condition is referred to herein as treatment of a subject that is “at risk” of developing the condition. Accordingly, administration of a composition can be performed before, during, or after the occurrence of the conditions described herein. Treatment initiated after the development of a condition may result in decreasing the severity of the symptoms of one of the conditions, or completely removing the symptoms. Non-limiting examples of subjects particularly suited to receiving the composition are those who may be exposed to natural or artificial UV irradation, individuals having genetic deficiencies in polypeptides involved in DNA repair (for instance, those suffering from xeroderma pigmentosum), and individuals who are immunosuppressed due to disease states (such as acquired immunodeficiency syndrome) or transplantation.

A composition that is introduced to a cell, including introduced to a subject that has or is at risk of developing a condition described herein, includes an effective amount of a PDG including a targeting sequence and PTD. As used herein, an “effective amount” is an amount effective to decrease or prevent (for prophylactic treatment) in a subject the symptoms associated with a condition described herein. In one embodiment, the composition further includes a pharmaceutically acceptable carrier. In one embodiment, the composition is administered to the subject by topical administration.

An aspect of the current disclosure is directed to a method for increasing the repair rate of damaged bases in a cell, preferably a skin cell. Similarly, another aspect of the disclosure is directed to a method for increasing UV-resistance in a wild-type cell. The methods include introducing to a cell exposed to or at risk of exposure to an agent that damages DNA a composition that includes an effective amount of a composition including a PDG polypeptide. The symptoms of this condition include, for instance, the increased presence of damaged DNA, increased mutagenesis rates, increased immunosuppression, increased tumor formation (for instance, increased actinic keratosis, increased basal cell carcinoma, and increased squamous cell carcinoma, and possibly increased melanoma), and increased incidence of apoptotic cells.

Whether the repair rate of damaged bases in a cell is increased can be determined by, for instance, assaying for the amount of damaged DNA in cells using a variety of techniques including coding sequence-specific repair assays (Bohr et al., Cell 40:359-369, 1985), and photoproduct removal as determined by ELISA assays using antibodies directed against cis-syn dimers (Clarkson et al., Mutation Res. 112:287-299, 1983). Alternatively, when assaying mitochondrially-targeted PDGs in human cells, the removal of lesions can be assayed by quantitative PCR assay that is specific for human DNA (see Ballinger et al., Exp. Eye Res. 68:765-772, 1999; and Ballinger et al., Circ. Res. 86: 960-966, 2000). For instance, cells exposed to an agent that damages DNA, such as UV light, can be treated with a composition including a polypeptide of the present disclosure. After a period of time sufficient to allow repair, the amount of damaged DNA in the cells can be determined and compared to the same type of cell that has not treated with the polypeptide. The presence of less damaged DNA in the cell treated with the polypeptide relative to the cell not treated indicates the polypeptide increases the repair rate of DNA. The repair rate of damaged DNA in in vivo cells may also be determined. For instance, an animal can be exposed to an agent that damages DNA, and treated with a composition including a polypeptide of the present disclosure. After a period of time sufficient to allow repair, skin biopsies are prepared and the amount of damaged DNA determined and compared to skin biopsies obtained from animals not treated with the polypeptide. The presence of less damaged DNA in cells in the biopsies treated with the polypeptide relative to cells in the biopsies not treated indicates the polypeptide increases the repair rate of DNA. Commonly accepted in vivo models are available for testing whether a polypeptide will increase the repair rate of DNA (for human models, see, for instance, Yarosh et al., Photochem. Photobiol. 69:136-140, 1999; for animal models, see, for instance, Mitchell et al., J. Invest. Dermatol. 95: 55-59, 1990).

The present disclosure further provides methods for treating mutagenesis in a cell, such as a skin cell, in response to an agent that damages DNA, such as UV light. In this aspect of the disclosure, mutagenesis rates are decreased. Mutagenesis results when repair of damaged DNA does not occur and, upon replication of the DNA, a different base is inserted. The method includes introducing to a skin cell exposed to or at risk of exposure to an agent that damages DNA, a composition that includes an effective amount of a PDG polypeptide. Whether the rate of mutagenesis in a cell is reduced can be determined by, for instance, by hprt mutagenesis assays (O'Neill et al., Mutat. Res. 45:103-109, 1977). Briefly, the measurement of mutagenesis using an hprt assay involves the selection of mammalian cells that are resistant to the killing effects of 6-thioguanine through a mutation in the hprt coding sequence. The assay relies on an inability of hprt-cells to activate 6-thioguanine for incorporation into DNA that results in cell killing. All cells with wild-type hprt are killed upon 6-thioguanine selection. The cells can be in vivo or ex vivo. The rate of mutagenesis in cells treated with a polypeptide of the present disclosure can be determined and compared to the rate of mutagenesis in cells not treated. The presence of a lower mutagenesis rate in treated cells relative to untreated cells indicates the polypeptide decreases the mutagenesis rate of DNA.

Also provided by the present disclosure are methods for treating immunosuppression in a cell, such as a skin cell, in response to an agent that damages DNA. The presence of damaged DNA results in a temporary, reversible immunosuppression. The method includes introducing to a skin cell exposed to or at risk of exposure to an agent that damages DNA, a composition that includes an effective amount of a PDG polypeptide. Whether immunosuppression in response to a DNA damaging agent is decreased can be determined by, for instance, measuring the transcription and/or translation of coding sequences that promote immunosuppression in response to a DNA damaging agent. For instance, the transcription and/or translation of a coding sequence encoding interleukin-10 (IL-10) or tumor necrosis factor alpha (TNF-α) can be measured using Northern blot analyses or commercially available antibody kits. The immunosuppression in cells treated with a polypeptide of the present invention can be determined and compared to the immunosuppression in cells not treated. The presence of higher levels of IL-10 and/or TNF-α in treated cells relative to untreated cells indicates the polypeptide decreases the immunosuppression of a cell in response to agents that damage DNA.

The present disclosure is also directed to methods for treating tumor formation in a cell in response to an agent that damages DNA. In this aspect of the disclosure, tumor formation is decreased. The types of tumors that may occur in response to an agent that damages DNA include actinic keratosis, basal cell carcinoma, squamous cell carcinoma, and melanoma. The method includes introducing to a skin cell that is at risk of developing a tumor in response to an agent that damages DNA, a composition that includes an effective amount of a PDG polypeptide. Cells at risk of developing a tumor in response to an agent that damages DNA include cells exposed to or at risk of exposure to an agent that damages DNA. Whether the formation of tumors in an animal is reduced can be determined by the use of animal models, for instance mice that have been exposed to solar-simulated light or exposure to sunlight. Solar-simulated light is light having a spectral profile which is similar to natural solar irradiation, i.e. the emission spectrum of a solar simulator looks similar to spectrum of a solar noon day. Wavelengths of light include ˜295-400 nm so is inclusive of UVA, UVB but not UVC which does not get through the ozone (see, for instance, Yoon et al., J. Mol. Biol. 299:681-693, 2000). The presence of a tumor can be determined by methods known in the art, and typically include cytological and morphological evaluation. The cells can be in vivo or ex vivo, including obtained from a biopsy. The rate of tumor formation in cells treated with a polypeptide of the present invention can be determined and compared to the rate of mutagenesis in cells not treated. The presence of lower rates of tumor formation in treated cells relative to untreated cells indicates the polypeptide decreases tumor formation.

Another aspect of the present disclosure is directed to treating the formation of apoptotic cells in response to an agent that damages DNA. Apoptotic cells are cells undergoing, or that have undergone, programmed cell death. In this aspect of the disclosure, the formation of apoptotic cells is decreased. The method includes introducing to a skin cell exposed to or at risk of exposure to an agent that damages DNA, a composition that includes an effective amount of a PDG polypeptide. Whether the formation of apoptotic cells is reduced can be determined by, for instance, using assays that detect apoptotic cells. Such assays include immunohistochemistry using antibodies against apoptotic-specific polypeptides associated with apoptotic cells, including, for instance, anti-caspase 8, anti-procaspase 9, and anti-G3PDH antibodies. Such antibodies are known to the art, and are available from, for instance, Trevigen (Gaithersberg, Md.) and Sigma-Aldrich, Co. (St. Louis, Mo.). The cells can be in vivo or ex vivo, including obtained from a biopsy. The formation of apoptotic cells in cells treated with a polypeptide of the present disclosure can be determined and compared to the formation of apoptotic cells in untreated cells. The presence of a lower apoptosis rate in treated cells relative to untreated cells indicates the polypeptide decreases the formation of apoptotic cells.

Also provided by the present disclosure are methods for treating psoriasis. The method can include introducing to a skin cell having or at risk of developing psoriasis, a composition that includes an effective amount of a PDG polypeptide. For example, an agent including one of the disclosed PDG polypeptides is administered to a subject, such as by a topical lotion, to treat psoriasis or a sign or symptom associated with psoriasis. The reduction or suppression a sign or symptom associated with psoriasis can be determined by comparing cells treated with a polypeptide of the present invention to cells not treated. Reducing or suppressing a sign or symptom associated with psoriasis is evidenced, for example, by a reduction in severity of some or all clinical symptoms associated with psoriasis, such as a reduction in psoriatic plaques, a reduction or prevention of joint inflammation, a reduction or prevention of the spreading of psoriasis, a reduction in the number of relapses of psoriasis, an improvement in the overall health or well-being of the subject, or by other parameters well known in the art.

In each aspect of the present disclosure, the PDG polypeptide optionally comprises a targeting sequence, such as a NLS or MLS, and/or a PTD, such as the TAT peptide.

The following examples are provided to illustrate certain particular features and/or embodiments. These examples should not be construed to limit the disclosure to the particular features or embodiments described.

### EXAMPLES

### Example 1

### Use of PDG for Therapeutic Applications

This example illustrates the use of PDG polypeptides for enhancing UV resistance.

Over a million new cases of skin cancer are diagnosed in the United States each year. Analyses of these tumors reveal frequent tandem mutations at dipyrimidine sites implicating cyclobutane pyrimidine dimers (CPDs) and 6-4 photoproducts as the initiating DNA lesions. Since repair of these lesions in mammalian cells is limited to NER, strategies have been implemented to augment the overall repair capacity in cells by activating BER via the introduction of PDGs. Clinical trials using T4-PDG in XP patients resulted in decreased actinic keratoses and cancers. However, when T4-PDG is introduced into normal mammalian cells and the cells are UV irradiated, survival decreases. It is believed that this is a result of T4-PDG-induced cytotoxic double-strand breaks at sites of CPDs in close proximity in complementary strands. In order to minimize the frequency of these T4-PDG-catalyzed double-strand breaks, as described herein, T4-PDG was genetically engineered such that it remains catalytically active, but incises CPDs randomly, rather than in clusters, by destabilizing the pre-catalytic steps of DNA bending and nucleotide flipping.

### Use of T4-PDG in the Prevention of Nonmelanoma Skin Cancer

There are clear and undisputable links between sunlight exposure and nonmelanoma skin cancers. In order to implement proactive strategies both to reduce the rates of nonmelanoma skin cancers and to delay the average age of disease onset, strategies have been developed to activate the BER pathway in mammalian cells for the removal of CPDs by introducing dimer-specific DNA glycosylases. The topical introduction of these enzymes into human skin cells increases the rate at which sunlight-induced DNA damages are removed, thus decreasing premalignant and malignant lesions and preventing UV-induced immunosuppression.

To add insights into amino acid residues and domains that are involved in the pre-catalytic steps of DNA bending and nucleotide flipping as well as to determine the architecture of the active site during catalysis, the co-crystal structure of T4-PDG was determined. These studies demonstrated T4-PDG was trapped as a reduced covalent imine intermediate with abasic (AP) site-containing DNA. Comparisons of co-crystal structures of T4-PDG revealed movements of the DNA and key amino acid side chains of T4-PDG that appear to be necessary to transition from a pre-catalytic complex to a post glycosylase complex. Thus, based on this structure, a series of studies have been developed to allow the determination of the role of specific amino acids in initiating and guiding the rearrangements in DNA to achieve a stable catalytically competent complex.

The disclosed PDG polypeptides can be characterized for their ability to bind, bend, flip and incise lesion-containing DNAs. In addition, molecular dynamics simulations of DNA flipping and catalysis can be carried out to provide a sequential blueprint for the T4-PDG reaction pathway.

The co-crystal structure of T4-PDG covalently linked with DNA containing an AP site reveals conformational differences with the precatalytic complex of T4-PDG E23Q and CPD-containing DNA. Many of these differences suggest a continuum of residue motions that are required to stabilize the catalytic complex. Therefore, it is predicted that there are three major sites within the enzyme-DNA interface that all contribute to the initiation, progression and stabilization of the nucleotide flipping: amino acid side chain interactions that alter the structure of the 1) complementary DNA strand 2) substrate DNA strand and 3) extrahelical base pocket. It is also predicted that the ability to achieve a catalytically competent enzyme-DNA complex will be significantly affected by the relative ease with which a sugar or nucleotide can be moved to an extrahelical position.

i. Amino acid side chains that bind to the complementary strand. The X-ray co-crystal structure of T4-PDG revealed that Arg22 and Arg26 occupy the intrahelical space vacated by the extruded Ade and that specific hydrogen bond networks are formed with the phosphates in the complementary strand backbone (Golan et al., J. Mol. Biol. 362:241-58, 2006). Previously, Arg26 has been suggested to be involved in nontarget DNA binding in which charge neutralization resulted in an active enzyme that had lost processive nicking activity (Dowd and Lloyd, J. Biol. Chem. 265(6):3424-31, 1990).

It is predicted that Arg22 and Arg26 act in a push and bind mechanism to force the nucleotide to its full 180° rotation through the major groove. It is also predicted that the activities of T4-PDG proteins will be differentially affected by the various templates proposed depending on whether the T4-PDG protein contains an individual mutation at Arg22 and Arg26, or a double mutation of Arg22 and Arg26. For those duplex DNAs in which there are no, or little, base stacking interactions, it is predicted that the identity of these side chains will be of minimal consequence on catalysis or binding. However, the purine and purine-modified bases are predicted to significantly decrease the efficiency of both parameters relative to WT enzyme. Specifically, it is predicted to change these arginine residues to lysine or histidine for charge conservation; glutamine for size conservation; and alanine for loss in size and charge of the side chain. These mutants will be made on both a WT and an E23Q T4-PDG background by site-directed mutagenesis using bidirectional PCR, followed by complete gene sequence confirmation. Mutated enzymes are also routinely purified to homogeneity using the New England BioLabs intein-chitin binding domain fusion and cleavage system (New England Biolabs).

Site-specific mutants of T4-pdg (Arg22 to Gln22 and Arg26 to Gln26) have been constructed and the enzymes purified to near homogeneity. As predicted from the above hypotheses, these mutant enzymes retain both the DNA glycosylase and AP lyase activities but have lost the processive nicking activity. The Gln22 T4-PDG confers a very modest level of enhanced survival to DNA repair- and recombination-deficient E. coli cells following irradiation with UV light. These mutants are predicted to decrease UV-induced cytotoxicity in human cells.

ii. Amino acid side chains that bind to the damage-containing strand. In addition to specific enzyme-DNA contacts being made in the complementary strand, the co-crystal structures have shown a limited number of hydrogen bond interactions with the damage-containing strand. It is predicted that these may be important to DNA bending, a process that is predicted to reduce the energy bather to flipping. Specifically, Arg3 will be mutated to lysine, histidine, glutamine, and alanine and assayed for changes in catalytic efficiency and DNA binding. The present co-crystal structure reveals that Arg3 makes a series of electrostatic interactions with phosphates surrounding the trapped reduced imine ring-opened sugar. Since Arg3 is absolutely conserved in all sequence homologs and paralogs of T4-PDG, it is predicted that these mutations will significantly reduce catalytic activity and binding when the base opposite the AP site is well stacked within the helix, while DNA substrates that readily flip the opposite base or sugar analog will be more tolerant of changes at Arg3. An additional residue that has undergone large conformational changes when compared between the two co-crystal structures is Arg117. While Arg3 interacts with both the +1 and −1 phosphates around the AP site, Arg117 is predicted to also be critical, since its large movement in the reduced complex shows direct interaction with the +1 phosphate adjacent to the abasic site. This residue is 100% conserved in phylogenetic analyses. Arg3 and Arg117 will be mutated to Lys, His, Gln and Ala and assayed as described above.

Site-specific mutants of T4-pdg (Arg3 to Lys3 and Arg117 to Gln117) have been constructed and the enzymes purified to near homogeneity. As predicted from the above hypotheses, these mutant enzymes retain both the DNA glycosylase and AP lyase activities but have lost the processive nicking activity. Both the Lys3 and Gln117 T4-PDG confer a very modest level of enhanced survival to DNA repair- and recombination-deficient E. coli cells following irradiation with UV light. These mutants are predicted to decrease UV-induced cytotoxicity in human cells.

iii. Amino acid residues that bind the extrahelical sugar and nucleotide. The binding pocket for the extrahelical nucleotide has also undergone significant rearrangements from that described for the pre-catalytic complex and these alterations suggest a hand-off in the stabilization of the extrahelical base. It is predicted that the reason that the binding pocket is designed to weakly bind the extrahelical nucleotide is so that following catalysis, the extrahelical nucleotide can be released back into the helix, thus facilitating enzyme turnover. Further, it is predicted that weakening of the binding affinity in the pocket will result in a decrease of the reaction rate due to an inability to stabilize the extrahelical nucleotide; conversely engineering the pocket to contain a number of aromatic side chains could lead to stabilization of the extrahelical nucleotide and poorer enzyme turnover. The key amino acid side chains involved in stably maintaining the bound complex are Asp87 and Tyr21. The side chains of Gln71 and Gln91 occupied one face of the adenine base in the noncovalent structure; however, in the covalent structure, they have moved back to their original position in the free enzyme. These data suggest that along the reaction coordinant, Gln71 and Gln91 may stabilize the base, while the covalent structure reveals that they are moving to facilitate release of the base. Phylogenetic analyses have revealed that Gln71 is an infrequent residue in that position, while Tyr71 is represented about 70% of the time. Similarly, Gln91 is poorly conserved, while this position is most often a valine or an acidic acid residue (>70% combined). In contrast, Asp 87 is 100% conserved and Tyr21 is about 90% conserved as either Tyr or Trp. These data suggest that in most PDGs, aromatic stacking and hydrophobic forces stabilize the extrahelical nucleotide. Mutations will be made that maintain charge, aromaticity, and hydrophobicity while others will neutralize charge, lose aromaticity and become hydrophilic.

The expected outcomes of these studies are that the enzymes carrying mutations in the extrahelical pocket will not be altered in catalytic efficiency or binding when assayed on an abasic site opposite sugar analogs and pyrimidines. In contrast, purines and purine derivatives are predicted to have increased binding with the Q71Y and Q91V mutants, but overall catalytic efficiency may be decreased due to a stabilization of the product complex and decreased turnover of the enzymes. In contrast, mutants exchanging their essential characteristics (charge, hydrophobicity, etc.) will display decreased binding, catalysis and processivity.

### Assay Methods for Characterizing Mutants of T4-PDG

i. Activity. The activity of the WT T4-PDG and its nuclear-targeted form will be measured using a range of substrate concentrations and k_{cat} and Kₘ will be calculated. It is believed that as the hydrogen bonding potential begins to rise, the k_{cat}/Kₘ will decrease. An exception to this may be examples where the substrate DNA contains an AP site in one strand, and the complementary strand contains either a THF, pyrrolidine, or either of the bi(cyclo) hexane pseudosugars (all baseless sites). In these examples, the stacking forces of the duplex DNA may cause the surrounding nucleotides to collapse, extruding both baseless sugars. Similarly, NMR studies (Cuniasse et al., J. Mol. Biol. 213(2):303-14, 1990) have established that pyrimidines opposite abasic sites are in equilibrium between intra- and extrahelical locations, while purines and modified purines remain stacked within the helix (Barsky et al., Nucleic Acids Res. 28(13):2613-26, 2000; Cuniasse et al., J. Mol. Biol. 213(2):303-14, 1990; Hoehn et al., Nucleic Acids Res. 29(16):3413-23, 2001; Kalnik et al., Biochemistry 28(8):3373-83, 1989; Lin et al., Nucleic Acids Res. 26(10):2385-91, 1998). The 2′ and 3′ endo (South and North, respectively) bicyclohexane pseudosugars have been used successfully in analyzing intermediates in the flipping mechanism of the bacterial cytosine-5 DNA methyltransferase M. HhaI due to conformational constraints of these abasic sites (Horton et al., Nucleic Acids Res. 32(13):3877-86, 2004; Marquez et al., Nucleosides Nucleotides Nucleic Acids 20(4-7):451-9, 2001). In addition to the abasic site analogs and pyrimidines, purine and purine-adducted nucleotides will also be assayed, such as oligodeoxynucleotides containing multi-membered-ring structures (e.g., benzo[a]pyrene, benz[a]anthracene and 7-hydroxypropano dG (Chary et al., Nucleic Acids Res. 23(8):1398-405, 1995; McNees et al., J. Biol. Chem. 272(52):33211-9, 1997; Minko et al., J. Biol. Chem. 278(2):784-90, 2003)). The purines and modified planar purines are expected to occupy increasing volumes of intrahelical DNA space opposite an AP site and due to increased stacking interactions will be more difficult to move to an extrahelical position. Similar strategies of increasing the intrahelical volume of modified pyrene bases to study UDG flipping have been used (Jiang and Stivers, Biochemistry 41(37):11236-47, 2002; Jiang et al., Biochemistry 41(37):11248-54, 2002). In addition to measuring activities on synthetic oligonucleotides in which it is not possible to assay for changes in the processivity of the enzymes, plasmid-nicking assays using DNAs containing 10-25 CPDs per DNA molecule will be used.

ii. Binding. In addition to measuring the catalytic efficiencies of these enzymes, K_{D}s will be determined using a reduced AP site in which NaBH4 (100 mM) is introduced simultaneously with the addition of UDG to rapidly convert all AP sites to reduced AP sites that are then no longer catalytic substrates for any glycosylase/AP lyase. Both gel shift and fluorescence anisotropy assays, which have been used to measure these binding constants, yielded comparable results. Initial data collection will use quantitative gel shift analyses. Since the experiments described above use DNA that cannot be cleaved, it is also advantageous to collect binding data using the natural AP containing DNA with catalytically inactive, but binding-competent enzymes. For these studies, the E23Q mutant of T4-PDG will be used (Doi et al., Proc. Natl. Acad. Sci. U.S.A. 89(20):9420-4 1992; Hori et al., Proc. Natl. Acad. Sci. U.S.A. 89(20):9420-4, 1992; Manuel et al., J. Biol. Chem. 270(6):2652-61, 1995).

iii. Fluorescence analyses for nucleotide flipping and bending. All mutant and WT T4-PDG enzymes will be characterized for modulation of flipping and bending.

### Strategies to Enhance UV-Resistance in Wild-Type Mammalian Cells

WT T4-PDG is currently being tested in human clinical trials for the treatment of XP patients and immunosuppressed organ transplant recipients. However, there is currently no data on how the WT enzyme functions in normal human keratinocytes since all previous studies have been performed in transformed normal or repair-deficient fibroblasts. Thus far, all prior studies of WT T4-PDG in WT mammalian cells have demonstrated a decrease in cell survival following UV exposure when compared to WT controls.

T4-PDG is not the only DNA glycosylase-AP lyase known that decreases cell survival when expressed as a transgene in mammalian cells; overexpression of either hNTH1 or hOGG1 in human lymphoblastoid cells significantly decreased survival and increased mutagenesis following ionizing radiation (Yang et al., Mutat. Res. 568(1):121-8, 2004). Overexpression of these glycosylases increased the frequency of double-stranded breaks resulting in cytotoxicity. It is believed that the increased cytotoxicity observed in WT mammalian cells overexpressing T4-PDG may be caused by a clustering of incisions within a DNA domain that contains closely opposed CPDs.

When T4-PDG incises all CPDs in complementary strands within ˜15 bp, it produces two single-strand breaks at these sites, resulting in a double-strand break and increased cytotoxicity. The positive clinical effects observed in XP patients are likely due to enhanced survival of NER-deficient cells by addition of T4-PDG. However, clinical applications of this enzyme for repair in healthy human patients may lead to increased cytotoxicity as is observed in all repair-proficient mammalian cells.

The PDG mutants described herein are likely to display lower catalytic efficiencies and a significant decrease in the number of double-stranded breaks at closely opposed dimer sites. Thus, both WT and WT T4-PDG -NLS will be expressed in initiated human keratinocytes and their level of expression, enzyme activity, cytotoxicity, frequency of double-stranded breaks and mutagenesis following UV will be evaluated. Concomitantly, genes encoding mutant forms of T4-PDG that have altered catalytic and nucleotide flipping parameters will be stably integrated and assessed for changes in UV survival. Similar studies will be performed in repair-deficient fibroblasts derived from XPA cells.

Transfection and Stable Selection of O3C Keratinocytes and XPA Fibroblasts with Plasmids Expressing Versions of T4-PDG with Differing Flipping and Catalytic Properties

It is an object herein to identify a form of T4-PDG that can optimize cell survival and resistance to mutagenesis in keratinocytes. Thus, O3C cells, a keratinocyte line that was originally derived from murine epidermal cell strain 291, will be used in T4-PDG transfection studies. These cells have a stable number of chromosomes and are readily transfected and selected using reagents such as LipofectAMINE (Invitrogen, Carlsbad, Calif.) and G418 sulfate, respectively. These studies will also include XPA fibroblasts, which have previously been used in repair complementation studies.

Table 1 describes the initial set of control T4-PDGs and mutant enzymes whose genes will be stably integrated into the genome of the O3C keratinocytes and XPA fibroblasts. The properties of WT and mutant enzymes are described in Table 1, along with expected outcomes. The initial characterization will include WT T4 PDG, a nuclear-targeted form of WT T4-PDG and mutants that are glycosylase and AP lyase deficient (E23Q), glycosylase negative and AP lyase positive (T2P and/or E23D), and mutants having reduced catalytic activity due to defects in various stages in nucleotide flipping (R26Q, Y21S, and R3K).

Each of the control and mutated forms of the enzyme will be cloned into shuttle vector pcDNA3.1 for continuous expression of the T4-PDG gene under the control of the CMV early promoter. This vector can be selected for stable integration in the O3C keratinocytes by G418 sulfate. A minimum of five clones of each construct will be expanded and assayed as described in the following section. If expression of the CMV promoter produces excess enzyme, an alternative inducible promoter system can be used, such as the GENESWITCH™ system (Invitrogen, Carlsbad, Calif.). This vector system utilizes a strategy in which the expression plasmid is transcriptionally silent (adenovirus E1b promoter driving the T4-PDG constructs) until a hybrid regulatory protein (consisting of a Ga14 DNA binding domain, a transactivating domain p65 and a mifepristone (RU486) receptor ligand binding domain) is activated by addition of RU486 (Sigma-Aldrich, St. Louis, Mo.) to the cells. This expression system would give rigorous control over the expression of the T4-PDG.

### Steady-State and Regulated Expression of T4-PDG and Selected Mutants

Following clonal expansion of the O3C keratinocytes stably transfected with plasmids that either constitutively or inducibly express T4-PDG and mutants with varying alterations that modulate bending, flipping and catalysis, each cell line will be assayed for constitutive expression of the CMV promoter or induced expression of the RU486 regulated promoter. Enzymes will be assayed by Western blot analyses using polyclonal rabbit antibodies directed against the entire T4-PDG protein. Since there are no homologs to T4-PDG in any eukaryotic cell, detection of expression of this 16 kDa protein is not complicated by endogenous cross reactivity. On each Western blot, a standardized set of WT T4-PDGs will be run ranging from 5 to 150 ng of enzyme. Following primary and secondary antibody incubations, the relative amount of protein will be determined by chemiluminescence using a Licor imager.

In addition to Western blot analyses, whole cell extracts will be used to detect CPD-specific nicking activities using both form I DNA plasmids containing on average 10-25 CPDs per molecule, or synthetic oligodeoxynucleotides containing a site-specific CPD. Both assays are routine and all reagents are immediately available. The dimer-specific nicking activities are readily detected because the O3C cells do not contain UV-specific glycosylase/AP lyases and consequently, the incisions that are produced will be a unique signature of the enzymes being expressed. For O3C cells transfected with the basic plasmid or cells expressing E23Q T4-PDG, no activities are expected.

TABLE 1
Properties of WT and Mutant T4-PDG Enzymes

| Enzyme | Properties |
|---|---|
| WT T4-PDG | Full glycosylase/AP lyase |
| | activity on CPDs; high |
| | processive nicking activity. |
| WT T4-PDG- | Same as WT T4-PDG except it |
| NLS | will localize in the nucleus. |
| E23Q | Inactive glycosylase/AP lyase, |
| | but retains nucleotide flipping |
| | at CPD sites. |
| T2P or E23D | Inactive as a CPD glycosylase |
| | but active as an AP lyase. |
| R26Q | Reduced catalytic activity as a |
| | DNA glycosylase-AP lyase, |
| | probably due to an inability to |
| | stabilize the flipped nucleotide; |
| | random target site location of |
| | CPDs in DNA domains; does |
| | not produce an appreciable |
| | number of double-strand breaks |
| | even at closely opposed CPDs. |
| Y21S | Reduced catalytic efficiency |
| | due to a destabilization of the |
| | flipped complex because the |
| | nucleotide binding pocket is |
| | compromised. |
| R3K | Reduced catalytic efficiency |
| | due to improper phosphate |
| | contacts on CPD-containing |
| | strand, possibly resulting in an |
| | insufficient DNA bending since |
| | DNA bending is a prerequisite |
| | for catalysis. |

### Survival, Repair and Mutagenesis in Cells Expressing Various T4-PDGs Following UVB Exposure

i. Cell survival. Following establishment of cell lines expression WT and mutant T4-PDGs, but prior to carrying out clonogenic assays that measure a cell's proliferative capacity to form colonies of >50 cells, a rapid, high throughput spectrophotometer assay, measuring numbers of live cells following UVB challenge will be used. This assay, developed by Dojindo Molecular Technologies, Inc. relies on cellular dehydrogenase activity in live cells to convert a water soluble tetrazolium salt, W5T-8[2-(2-methoxy-4-nitrophenyl)-3-(4-nitrophenyl)-5-(2,4-disulfophenyl)-2H-tetrazolium] to a yellow-colored formazan that can be read in a 24 or 96-well plate reader. This “Cell Counting Kit-8” was specifically designed for chemical cytotoxicity assays and thus, is well-suited to the proposed investigations with UVB challenge. For cells harboring endogenously produced enzyme from the CMV promoter, no inductions are necessary. However, the kinetics of expression of the RU486-regulated expression system will be determined prior to UV challenge. Preliminary analyses have established a UVB-dose dependent response for the O3C keratinocytes to induce sufficient cell killing that can be readily detected, while not reducing survival so severely to interfere with measurements in the linear range of the assay (˜3 log sensitivity range). Additionally, conditions have been established for CPD immunostaining following UV irradiation of keratinocytes to allow for the detection of DNA repair. Both mylar filtered (wavelength cut off 313 nm) and unfiltered UVB light can be used. Results will be validated using a colony-forming assay as previously described in the Lloyd laboratory (Rinaldy et al., Proc. Natl. Acad. Sci. U.S.A. 87(17):6818-22, 1990).

ii. Histone H2AX phosphorylation as an independent measure of enzyme-induced double-stranded breaks. Phosphorylation of histone H2AX on Ser 139 (γH2AX) is well documented to occur in response to agents that produce double-stranded breaks (Rogakou et al., J. Biol. Chem. 273(10):5858-68, 1998) including ionizing radiation, topoisomerase I and II inhibitors, tobacco smoke condensate, and UV irradiation. Previous studies have demonstrated that phosphorylated H2AX is a specific and quantitative marker of double-stranded breaks in nuclear chromatin. Within minutes after ionizing radiation, γH2AX appears in chromatin as discrete foci that co-localize with other cell cycle checkpoint and repair proteins. Thus, quantitation of γH2AX after UV exposure in control O3C keratinocytes and XPA fibroblasts and those that express T4-PDG or compromised T4-PDGs will be a direct measure of enzyme-induced double-stranded breaks. Experimentally, cells will be grown to near confluence to minimize replication-associated direct double-stranded breaks. Cells will be harvested immediately prior to, and following, various UVB exposures, and at 30 min intervals for 2 hrs. These time points will measure double-stranded break formation at intervals on a much shorter time scale than would be associated with UV-induced apoptotic responses that require greater than 18 hours to manifest. The appearance and magnitude of the γH2AX response will be measured by Western blot analyses using anti-phosphohistone γH2AX clone JBWIO3 mouse monoclonal IgG (Upstate Biotechnology, Inc.). It is believed that γH2AX will significantly increase in O3C cells expressing WT T4-PDG shortly after UV-irradiation, while minimally increasing over control values in cells expressing forms of T4-PDG that are defective in nucleotide flipping or bending, in which there is a lack of clustered DNA incisions at CPD sites.

iii. Neutral comet assay. As previously described, there is increasing evidence suggesting that double-stranded breaks represent a major cytotoxic lesion following toxicant exposure. Thus, the frequency of double-stranded breaks in O3C cells expressing WT T4-PDG and various mutants will be assessed using a neutral microgel electrophoresis assay (Singh and Stephens, Mutat. Res. 383(2):167-175, 1997). To establish standard assay conditions for these cells, exponentially growing O3C cells will either be untreated or irradiated with UVB and allowed to grow 0-24 hr at 37° C. At each time, both irradiated and non-irradiated cells (˜3×10⁴ per slide) will be chilled to 4° C., embedded in high resolution agarose, lysed, and sequentially treated with RNase A and proteinase K. DNA within the lysed cells will be subjected to an electric field, causing the DNA containing double-stranded breaks to migrate from the main focus of DNA. Following visualization with YOYO1, random fields of cells will be visualized by fluorescent microscopy and quantitated using software associated with the camera. It is anticipated that a minimum of 100 cells each will be quantitated for assessments of relative double-stranded breaks.

Having established reproducible conditions for this neutral comet assay using the control O3C cells, formation of double-stranded breaks as a function of UVB-dose and repair time, will be measured in cells expressing T4-PDG and its mutants. It is predicted that UV-irradiated cells that express WT T4-PDG will contain a significantly higher frequency of enzyme-induced double-stranded breaks than irradiated controls or cells expressing catalytically active, but flipping or bending compromised enzymes.

iv. Mutational analyses using a shuttle vector. An experimental strategy that has been used extensively to analyze mutagenic events relies on the replication of a damaged shuttle vector in WT and repair-deficient cells, and a subsequent evaluation of mutagenic events using colorimetric analyses in an E. coli system (Kraemer and Seidman, Mutat Res. 220(2-3):61-72. 1989; Seidman et al., Gene 38(1-3):233-7 1985). This vector contains: 1) the supF tyrosyl suppressor tRNA gene (the mutagenic target); 2) the pBR327 origin of replication for bacterial replication; 3) ampicillin resistance gene; β-lactamase for bacterial selection; and 4) the bidirectional origin of replication of SV40. The supF gene, which is subsequently scored in E. coli using a blue/white screening assay, is exceptionally well suited for defining mutational spectra because 96% of all possible base changes and all deletions will result in the inactivation or decreased function of the tRNA.

To evaluate mutagenic frequencies and spectra, pZ189 will be irradiated with UVB to produce varying numbers of CPDs per DNA molecule. The extent of modification can be determined by the conversion of form Ito form II DNAs to nicked molecules by treatment with WT T4-PDG, in which the average number of lesions per plasmid is calculated by the −ln of the surviving mass fraction of form I DNA. Target theory can then be used to extrapolate to the approximate number of lesions per tRNA gene. Control and damaged DNAs (about 1 μg) will be transfected into O3C cells expressing various T4-PDGs or control cells. After 36 hr, replicated plasmid DNAs will be recovered as a Hirt supernatant and digested with Dpn Ito remove nonreplicated DNAs. E. coli cells, MBM7070, will be transformed with an aliquot of the digested Hirt supernatant DNAs. Transformed cells will be selected with 50 μg/ml ampicillin and scored by cleavage of 5-bromo-4-chloro-3-indol, β-D galactoside after isopropyl-1-thio-β-D-galactoside (IPTG) induction. Colonies that are white or light blue will be replated, plasmid DNAs isolated, and the tRNA gene sequenced. It is anticipated that there will be increases in the frequency of mutations when the damaged plasmids are replicated through the control cells relative to that observed for cells expressing catalytically competent T4-PDGs.

v. Genomic DNA mutation frequencies at the tk locus. Since mutagenic analyses using shuttle vectors measures mutation frequencies without the DNA being in a fully chromatin-associated state, it is also proposed to measure induced mutation frequencies at the tk locus. If spontaneous tk mutations are high in O3C cells, these cells can be passaged in CHAT media (10 μM deoxycytidine, 200 μM hypoxanthine, 0.2 μM aminopterine and 20 μM thymidine) to reduce this frequency prior to UV irradiation. Control and O3C cells expressing various T4 PDGs will be UV-irradiated or left untreated and after about 3 days plated in 96-well dishes at ˜4×10⁴ cells per well in a selection media containing 2 μg/ml trifluorothymidine. Cells that become tk⁻ will be assayed approximately 2 weeks following selection. Mutation frequencies will be determined by plating efficiency of selected versus nonselected cells. It is anticipated that mutation frequencies at the tk locus can be decreased in flipping-compromised mutants due to a lack of induction of double-stranded breaks.

### Example 2

### PDG-TAT Fusion Proteins

This example describes methods for producing PDG-TAT fusion proteins that can be used to deliver PDG polypeptides into the epidermal and dermal skin layers without the need of liposomes. In one example, the disclosed proteins are used to treat, such as reduce or eliminate, skin cancers.

Fusion of a PTD of HIV TAT polypeptide to the carboxy-terminal region of PDG will facilitate the efficient delivery of active DNA repair proteins to the skin. This will allow a mechanism for the delivery of proteins into the epidermal and dermal skin layers and will have the capacity to reduce or eliminate skin cancers.

Given the potential of the TAT polypeptide to deliver biologically active enzymes in organisms, the fusion of the TAT polypeptide to a PDG that is targeted to either the nucleus or mitochondria, when applied to the outer portion of the skin (stratum corneum), will penetrate through this layer of dead cells and distribute to all cell types in the epidermal and dermal layers. Once inside the cells, the PDG will localize to the appropriate intracellular organelle and initiate repair of CPDs and other oxidative DNA lesions. This will serve to both minimize mutations that can lead to cancer and prevent UV-induced immunosuppression, thus allowing the natural immune system to kill emerging cancer cells.

i. Plasmid Construction. In order to produce proteins that can be delivered to cells, either in tissue culture or into skin, protein expression systems are designed that produce large quantities of the desired proteins. Since there are many different types of PDGs that can both localize to different portions of the cell and be detected by different methods, a plasmid DNA molecule (pET22b) was constructed that would contain a strong E. coli T7 promoter, a series of DNA restriction sites, the DNA encoding the 11 amino acid TAT polypeptide, and a 6-His-affinity tag; this plasmid will be here-after referred to as the “universal acceptor TAT plasmid.”

The following plasmid constructs for the expression of various PDGs were produced: CV-PDG-pET22b; CV-PDG-nls2-pET22b; CV-PDG-GFP-pET22b; CV-PDG-nls2-GFP-pET22b; and MLS-CV-PDG-pEGFP (T4-PDG=bacteriophage T4 pyrimidine dimer glycosylase; CV-PDG=Chlorella virus encoded pyrimidine dimer glycosylase; NLS1=nuclear localization sequence 1 (PKKRKRRL; SEQ ID NO: 11); NLS2=nuclear localization sequence 2 (PKKKRKRL; SEQ ID NO: 12); MTS29=mitochondrial targeting sequence (MGVFCLGFWGLGRKLRTFGKGPLQLLSRL; SEQ ID NO: 15), derived from the uracil DNA glycosylase mitochondrial targeting sequence; EGFP=enhanced green fluorescent protein used for microscopic visualization; and His6=a DNA sequence encoding six consecutive histidines).

Synthetic deoxyoligonucleotides were designed to amplify the respective gene constructs from each plasmid. The sequences of the forward and reverse primers are shown in Table 2 and Table 3, respectively.

TABLE 2
Forward primers

| | | | SEQ ID |
|---|---|---|---|
| Primer | Name | Sequence | NO: |
| A | CV-PDG Nde I | cttgcccatatgacacgtgtgaa | 16 |
| | | tctcgta | |
| B | MLS35 CV-PDG | cttgcccatatgggcgtgttttg | 17 |
| | Nde I | cttaggc | |
| C | Delta 6 MLS35 | cagttattatcgcgcttaacacg | 18 |
| | | tgtgaatctcgta | |
| D | NLS 1 GFP pET | Ccaaagaagaggaaaaggaggct | 19 |
| | FWD | aggatccatcgccacc | |
| E | NLS 1 pET22b | Ccaaagaagaggaaaaggaggct | 20 |
| | FWD | aaagctttatggccgc | |

TABLE 3
Reverse primers

| | | | SEQ ID |
|---|---|---|---|
| Primer | Name | Sequence | NO: |
| F | CV-PDG | gcggccgctaagcttattattg | 21 |
| | | ctggttttagcttt | |
| G | CV-PDG NLS2 | gcggccgctaagctttagcctc | 22 |
| | | ttcctctttttctt | |
| H | CV-PDG GFP | gcggccgctaagcttgtacagc | 23 |
| | | tcgtccatgcc | |
| I | Delta 6 MLS35 | tacgagattcacacgtgttaag | 24 |
| | | ccggataataactg | |
| J | NLS 1 pET22b | tagcctccttttcctcttcttt | 25 |
| | | ggcccgggattattgct | |

PCR reaction preparations included 5 μl of 10× reaction buffer (Pfu Turbo), 10 ng of dsDNA template, 125 ng of oligonucleotide forward primer, 125 ng of oligonucleotide reverse primer, 1 μl (10 mM) of dNTP master mix and double-distilled water to a final volume of 50 μl. One μl of Pfu Turbo DNA polymerase (2.5 U/μl) was added to the reaction mixture and DNA was amplified by PCR. PCR conditions included performing 28 or 22 cycles, each cycle including three steps of 94° C. for 30 seconds; 62° C. for 30 seconds; and 68° C. for 3 or 8 minutes (3 minutes for fragment amplification and 8 minutes for plasmid or site directed mutagenesis). Twenty-eight cycles of PCR were performed for fragment amplification and 22 cycles for plasmid or site directed mutagenesis.

Following PCR, each DNA fragment was digested with NdeI and HindIII, the DNAs purified from contaminants by electrophoresis through 1% agarose gels and extraction and purification from the gel. Concomitantly, the universal acceptor TAT vector was digested with both NdeI and HindIII. The DNA fragments were then ligated into the TAT vector. Ligation reaction preparations were carried out at 16° C. overnight and included 7 μl of PCR amplified fragment; 2 μl of digested TAT-pET22b vector; 1 μl of 10×T4 DNA Ligase Buffer (NEB); and 0.25 μl of T4 DNA Ligase. BL 21 E. coli chemically competent cells were obtained from Invitrogen (Carlsbad, Calif.) and colonies were selected on LB agar plates by 50 μg/ml ampicillin at 37° C. Individual colonies were randomly selected, and plasmid DNAs were isolated and analyzed for correct inserts by restriction digestion with NdeI and HindIII. Plasmids containing inserts of appropriate length were further analyzed by DNA sequencing. Only those plasmids with the correct sequence were further analyzed for protein expression. The TAT constructs were as follows: CV-PDG-TAT-pET22b (primers A and F); cv-NLS1-TAT-pET22b (primers E and J; site directed mutagenesis of plasmid 8); cv-NLS2-TAT-pET22b (primers A and G); cv-EGFP-TAT-pET22b (primers A and H); cv-NLS1-EGFP-TAT-pET22b (primers E and J; site directed mutagenesis of plasmid 11); cv-NLS2-EGFP-TAT-pET22b (primers A and H); MLS(35)-CV-PDG-TAT-pET22b (primers B and F); MLS(35)-CV-PDG-EGFP-TAT-pET22b (primers B and H); MLS(29)-CV-PDG-TAT-pET22b (primers C and I; site directed mutagenesis of plasmid 12); and MLS(29)-CV-PDG-EGFP-TAT-pET22b (primers C and I; site directed mutagenesis of plasmid 13).

ii. Protein Expression and Purification. The plasmids listed immediately above were transformed into E. coli and selected for ampicillin resistance. Individual colonies were randomly selected and grown in LB to an optical density of 0.6 at O.D. 600 and protein expression induced by the addition of IPTG to a final concentration of 0.5 mM for 4 hrs at 18° C. Cells were harvested by centrifugation at 3000×g in a Sorval GSA rotor at 4° C. for 10 min. Cells were resuspended in 50 mM sodium phosphate (pH 7.0), 300 mM NaCl, 10 mM Imidazole, and protease inhibitors; lysed by passage through a French Pressure Cell at 11,000 psi; and cell debris removed by centrifugation at 10,000×g in a Sorval GSA rotor for 20 min at 4° C. Soluble proteins were bound to a His 6-affinity matrix, Talon Metal Affinity Resin via batch binding. The matrix was washed twice with 20 bed volumes of the resuspension buffer and then transferred to a 25 ml column. The column was washed with 5 bed volumes of resuspension buffer and subsequently eluted with an imidazole gradient (250 mM). Column fractions were collected and analyzed for purity by SDS polyacrylamide gel electrophoresis.

iii. PDG-TAT Glycosylase Activity Assay. To show that the presence of the TAT polypeptide did not alter the activity of the enzyme, a plasmid nicking assay was performed using both PDG-TAT and wild type PDG. Plasmid was irradiated with UVC at 100 μW/cm² for 4.5 min. to induce TT-dimers. PDG enzyme was diluted into 1×PDG buffer (40 mM Hepes, pH 7.0, 75 mM KCl, 0.5 mM DTT, and 20 mM EDTA pH 8.0), mixed with UVC irradiated plasmid, and incubated for 1 hr at 37° C. The enzyme was heat inactivated at 100° C. for 20 min, and the plasmid products were separated by electrophoresis on a 0.8% agarose gel. The results showed that the TAT polypeptide has no effect on the activity of the PDG.

iv. TAT-mediated Protein Transduction in Cells in Culture. HeLa fibroblast cells and O3C keratinocytes were plated in 12-well culture dishes and allowed to grow to 70% confluence. Fifty μg purified PDG-TAT protein/ml of media was delivered to the cells and cells were incubated for 6 hr. Cells were trypsinized, re-plated on microscope coverslips, and allowed to adhere to the coverslip for 6 hr. Cells were then fixed in 4% formaldehyde, washed with PBS, and mounted on a glass slide using a SlowFade Antifade Kit (Molecular Probes, Eugene, Oreg.). Localization of purified PDG-TAT protein was determined by fluorescence microscopy.

v. TAT-fused PDG Glycosylase Activity to Mediate Repair of TT Dimers in Cells in Culture. O3C Keratinocytes were plated in 100 mm tissue culture dishes and allowed to grow to about 70% confluence. Cells were treated with 100 J/m² UVB and either incubated with 50 μg purified PDG-TAT/ml of media or left without glycosylase treatment. At intervals of 0, 2, 4, and 6 hr following UVB irradiation, DNA was isolated from the cells according to the Qiagen DNeasy Tissue Kit Protocol (Qiagen). An enzyme-linked immunosorbent assay (ELISA) was used to quantify the number of TT dimers present in the cells following UV irradiation with or without incubation with the PDG-TAT. The 96-well ELISA plates were first coated with 1% protamine sulphate (Sigma-Aldrich) solution and incubated at 37° C. until dry. Serial dilutions of DNA harvested from the keratinocytes were subsequently plated into the pre-coated plate and allowed to dry overnight at 37° C. The plates were washed with PBS+0.05% Tween-20 then blocked with PBS+2% fetal bovine serum (FBS) for 30 min at 37° C. Following washing with PBS, 100 μl Dwell of TT dimer antibody (Kamiya Biomedical Company diluted 1:500 with PBS+Tween-20) was incubated for 30 min at 37° C. Following washing with PBS, 100 μl/well of peroxidase-goat anti-mouse antibody (Zymed Laboratories diluted 1:3000 with PBS+Tween-20) was incubated for 30 min at 37° C. After 3 washes with PBS+Tween-20, and 2 washes with citrate phosphate buffer (pH 5.0), 100 μl/well of substrate solution (0.04% o-phenylene diamine, 0.007% H₂O₂ in citrate-phosphate buffer) was added and incubated for 30 min at 37° C. Fifty μl/well of 2M H₂SO₄ was added to stop the reaction and the absorbance was measured at 490 nm using a 96-well plate reader (Spectra Max 190, Molecular Devices).

### Example 3

### Evaluation of PDG Polypeptides in a Reconstituted Skin Model

A reconstituted skin model can be used to test whether the PDG polypeptides are efficiently delivered to skin cells and localize to the appropriate organelle. The MatTek (Ashland, Mass.) EPIDERM FT™ skin model has previously been used to assess cellular responses to UV exposure. EPIDERM FT™ skin samples are induced to form a stratum corneum according to the manufacturer's instructions. Samples are either untreated or treated with one of the PDG polypeptides provided herein. After polypeptide treatment, such as after about 30 min, the skin samples are either unexposed or exposed to UV light. In one example, some samples are processed immediately following UV exposure, while others are processed after about 4 h to allow DNA repair to occur.

Methods of determining localization of the PDG polypeptide are well known in the art. For example, if the PDG polypeptide comprises a fluorescent tag, such as green fluorescent protein, the skin samples can be evaluated by immunofluorescence microscopy. Immunofluorescence microscopy can be performed by mounting vertical cryosections (5 μm) of the skin samples on slides with PROLONG GOLD™ (Invitrogen, Carlsbad, Calif.) antifade reagent and DAPI (Molecular Probes, Eugene, Oreg.), sealed and left to dry prior to examination. DAPI staining is used to show the location of the nucleus (blue); therefore, in this example, if the polypeptide localizes to the nucleus, there will be an overlap of green and blue fluorescence.

The reconstituted skin model can also be used to evaluate CPD repair. As described above, the skin samples are either treated or untreated with PDG polypeptide, then unexposed or exposed to UV irradiation. Samples are then processed for immunostaining using an antibody specific for CPD lesions. DAPI staining can be used to identify cell nuclei.

### Example 4

### SKH-1 Mouse Model

SKH-1 hairless mice can be used to evaluate CPD repair, sunburn cell formation (apoptosis), and carcinogenesis. SKH-1 mice are euthymic and immunocompetent, and are generally used for wound healing and dermal research. Various dilutions of PDG-containing liposomes are applied to 10 predetermined sites on the backs of SKH-1 mice. Mice treated with liposomes without enzyme serve as controls. To determine the effectiveness of delivery, treated mice are euthanized and punch biopsies are taken from each sector. To determine localization of CV-PDG-NLS, skin biopsies are flash frozen and sectioned for immunohistochemistry using antibodies reactive with CV-PDG. Alternatively, for liposomes containing the CV-PDG-NLS-EGFP fusion protein, punch biopsies are flash frozen, sectioned and directly visualized with fluorescent microscopy for distribution throughout the epidermis.

After at least 30 minutes, mice are irradiated with 20 kJ/m² (a 2× Mean Erythemal Dose) of UVB (Westinghouse FS20T12 sun lamps) and either immediately euthanized or euthanized after 4 or 12 hours of repair. Punch biopsies are taken from each of the 10 sectioned areas and fixed in a solution of 75% methanol and 25% acetic acid for 1 hour, followed by incubation in 70% ethanol for one hour and in 5% sucrose for an additional hour. The biopsies are frozen in liquid nitrogen until cryosectioning. Sections on slides are incubated in 70% ethanol, 0.07 N NaOH for 4 minutes and washed extensively in Tris-buffered saline. Following a 30 minute proteinase K treatment, tissues are incubated overnight with antibodies that are specific to the cyclobutane pyrimidine dimers, such as TDM2 (Mori et al., Photochem. Photobiol. 54(2):225-232, 1991). Following incubation with a fluorescent secondary antibody and DAPI, antibody reactivity is quantitated, and the kinetics of dimer removal evaluated.

The kinetics of removal of DNA lesions can also be measured. Liposomal lotions are applied to the backs of SKH-1 mice, with the upper torso receiving liposome alone, the mid-torso receiving liposome with T4-PDG and the lower torso receiving liposomes containing CV-PDG-NLS. After 30 minutes, mice receive 0, 5, 10, 20, or 40 kJ/m² UVB light (Westinghouse FS20T12 sun lamps). At each UVB dose, 2 mice are euthanized immediately or euthanized after 6 hours. Punch biopsies are taken and processed as described above for CPD quantitation. It is anticipated that mice receiving the 20 kJ/m² dose and liposome alone will have a significant number of dimers at both the 0 and the 6 hour repair timepoints, due to the relatively slow kinetics of dimer removal in mice. Portions of the back receiving CV-PDG-NLS will show significant, if not a near complete loss of dimers at 6 hours, even at the highest UV doses. The rate of repair in the nuclear-targeted forms of these enzymes will be significantly faster than that measured for the untargeted form of T4-PDG.

In order to assay for the appearance of sunburned apoptotic cells, the same skin punch biopsies are evaluated using the TUNEL assay (Fluorescein Apoptosis Detection System, Promega), which labels highly fragmented genomic DNAs. Skin biopsies are fixed overnight in freshly prepared, buffered 4% paraformaldehyde, washed in PBS and embedded in paraffin until analyses. Deparaffinned samples are treated as described in the manufacturer's protocol. Additionally, portions of the skin biopsies can be used for Western blot analyses to assay for biomarkers of UV-induced cellular stress. These can include the use of antibodies against, for example, IL-10, IL-4, PGE2, PAF, and p53.

Carcinogenesis studies can be performed using eight week-old female SKH-1 mice. Prior to UV irradiation, a liposomal lotion containing no enzyme, T4-PDG or CV-PDG-NLS is applied to the backs of the mice. After a minimum elapsed time of 30 minutes, mice are exposed (or left unexposed) to doses of UVB light 12K J/m² (for approximately 30 minutes) with Westinghouse FS20T12 sun lamps, 3 times per week for about 8-24 weeks. Measurements of approximate skin thickening are determined using a bi-fold skin assay (Vayalil et al., Carcinogenesis 24(5):927-936, 2003). This UV skin cancer induction protocol forms squamous cell carcinomas in SKH-1 hairless mice beginning after about 10-12 weeks (Balasubramanian et al., Oncogene 18(6):1297-1302, 1999; Katiyar et al., J. Natl. Cancer Inst. 89(8):556-66, 1997; Mitchell et al., Photochem. Photobiol. 73(1):83-9, 2001; Reagan-Shaw et al., Onocgene 23(30):5151-60, 2004; Vayalil et al., Carcinogenesis 24(5):927-36, 2003) Animals are routinely observed from the onset, and frequency and size of tumors recorded.

This disclosure provides PDG polypeptides and their methods of delivery and use. The disclosure further provides isolated polypeptides comprising the PDG polypeptides, a protein transduction domain and a targeting sequence. It will be apparent that the precise details of the methods described may be varied or modified without departing from the spirit of the described disclosure. We claim all such modifications and variations that fall within the scope and spirit of the claims below.

1. An isolated polypeptide, comprising:
a pyrimidine dimer-specific glycosylase (PDG) amino acid sequence selected from the group consisting of SEQ ID NO: 2, SEQ ID NO: 3, SEQ ID NO: 4, SEQ ID NO: 5, SEQ ID NO: 7, SEQ ID NO: 8, SEQ ID NO: 9 and SEQ ID NO: 10;
a nuclear or mitochondrial targeting sequence; and
a protein transduction domain (PTD).

2. The isolated polypeptide of claim 1, wherein the protein transduction domain comprises a human immunodeficiency virus (HIV) transactivator of transcription (TAT) peptide.

3. The isolated polypeptide of claim 2, wherein the TAT peptide comprises the sequence of SEQ ID NO: 40.

4. The isolated polypeptide of claim 1, wherein the nuclear or mitochondrial targeting sequence is a nuclear targeting sequence comprising the sequence of SEQ ID NO: 11.

5. The isolated polypeptide of claim 1, wherein the nuclear or mitochondrial targeting sequence is a nuclear targeting sequence comprising the sequence of SEQ ID NO: 12.

6. The isolated polypeptide of claim 1, wherein the nuclear or mitochondrial targeting sequence is a mitochondrial targeting sequence comprising the sequence of SEQ ID NO: 15.

7. The isolated polypeptide of claim 1, wherein the nuclear or mitochondrial targeting sequence is fused to the carboxy terminus of the PDG amino acid sequence.

8. The isolated polypeptide of claim 1, wherein the nuclear or mitochondrial targeting sequence is fused to the amino terminus of the PDG amino acid sequence.

9. The isolated polypeptide of claim 1, wherein the PTD is fused to the carboxy terminus of the PDG amino acid sequence.

10. A pharmaceutical composition comprising a therapeutically effective amount of the polypeptide of claim 1 in a pharmaceutically acceptable carrier.

11. An isolated polynucleotide encoding the polypeptide of claim 1.

12. A vector comprising the polynucleotide of claim 11.

13. An isolated cell comprising the polynucleotide of claim 11.

14. A method for increasing the repair rate of damaged bases in a cell, comprising:
contacting a cell in need of DNA repair with a therapeutically effective concentration of an agent comprising an isolated polypeptide of claim 1, thereby increasing the repair rate of damaged DNA in the cell compared to an untreated cell.

15. The method of claim 14, wherein the cell is a cancer cell.

16. The method of claim 15, wherein the cancer cell is a keratinocyte carcinoma.

17. The method of claim 14, wherein the cell is a skin cell.

18. (canceled)

19. A method for increasing the UV-resistance of a cell, comprising:
contacting the cell with an effective concentration of an agent comprising an isolated polypeptide of claim 1, thereby increasing the UV-resistance of the cell compared to an untreated cell.

20. A method of treating a skin disorder in a subject, comprising contacting the skin of the subject in need treatment with a therapeutically effective concentration of an agent comprising an isolated polypeptide of claim 1, wherein the skin disorder is selected from the group consisting of skin cancer, psoriasis and actinic keratosis.

21. A method of treating UV-induced immunosuppression in a subject, comprising contacting the skin of the subject in need treatment with a therapeutically effective concentration of an agent comprising an isolated polypeptide of claim 1.


SEQUENCE LISTING (43 sequences)

SEQ ID NO: 1 - protein, 138 aa, Bacteriophage T4
MTRINLTLVSELADQHLMAEYRELPRVFGAVRKHVANGKRVRDFKISPTFILGAGHVTFFYDKLEFLRKRQIELIAECLKRGFNIKDTTVQDISDIPQEFRGDYIPHEASIAISQARLDEKIAQRPTWYKYYGKAIYA

SEQ ID NO: 2 - protein, 138 aa, Bacteriophage T4
MTQINLTLVSELADQHLMAEYRELPRVFGAVRKHVANGKRVRDFKISPTFILGAGHVTFFYDKLEFLRKRQIELIAECLKRGFNIKDTTVQDISDIPQEFRGDYIPHEASIAISQARLDEKIAQRPTWYKYYGKAIYA

SEQ ID NO: 3 - protein, 138 aa, Bacteriophage T4
MTRINLTLVSELADQHLMAEYQELPRVFGAVRKHVANGKRVRDFKISPTFILGAGHVTFFYDKLEFLRKRQIELIAECLKRGFNIKDTTVQDISDIPQEFRGDYIPHEASIAISQARLDEKIAQRPTWYKYYGKAIYA

SEQ ID NO: 4 - protein, 138 aa, Bacteriophage T4
MTRINLTLVSELADQHLMAEYRELPQVFGAVRKHVANGKRVRDFKISPTFILGAGHVTFFYDKLEFLRKRQIELIAECLKRGFNIKDTTVQDISDIPQEFRGDYIPHEASIAISQARLDEKIAQRPTWYKYYGKAIYA

SEQ ID NO: 5 - protein, 138 aa, Bacteriophage T4
MTRINLTLVSELADQHLMAEYRELPRVFGAVRKHVANGKRVRDFKISPTFILGAGHVTFFYDKLEFLRKRQIELIAECLKRGFNIKDTTVQDISDIPQEFRGDYIPHEASIAISQAQLDEKIAQRPTWYKYYGKAIYA

SEQ ID NO: 6 - protein, 141 aa, Chlorella virus-1
MTRVNLVPVQELADQHLMAEFRELKMIPKALARSLRTQSSEKILKKIPSKFTLNTGHVLFFYDKGKYLQQRYDEIVVELVDRGYKINVDAKLDPDNVMTGEWYNDYTPTEDAFNIIRARIAEKIAMKPSFYRFTKAKTSNN

SEQ ID NO: 7 - protein, 141 aa, Chlorella virus-1
MTQVNLVPVQELADQHLMAEFRELKMIPKALARSLRTQSSEKILKKIPSKFTLNTGHVLFFYDKGKYLQQRYDEIVVELVDRGYKINVDAKLDPDNVMTGEWYNDYTPTEDAFNIIRARIAEKIAMKPSFYRFTKAKTSNN

SEQ ID NO: 8 - protein, 141 aa, Chlorella virus-1
MTRVNLVPVQELADQHLMAEFQELKMIPKALARSLRTQSSEKILKKIPSKFTLNTGHVLFFYDKGKYLQQRYDEIVVELVDRGYKINVDAKLDPDNVMTGEWYNDYTPTEDAFNIIRARIAEKIAMKPSFYRFTKAKTSNN

SEQ ID NO: 9 - protein, 141 aa, Chlorella virus-1
MTRVNLVPVQELADQHLMAEFRELKMIPKALARSLRTQSSEKILKKIPSKFTLNTGHVLFFYDKGKYLQQRYDEIVVELVDRGYKINVDAKLDPDNVMTGEWYNDYTPTEDAFNIIQARIAEKIAMKPSFYRFTKAKTSNN

SEQ ID NO: 10 - protein, 141 aa, Chlorella virus-1
MTRVNLVPVQELADQHLMAEFRELKMIPKALARSLRTQSSEKILKKIPSKFTLNTGHVLFFYDKGKYLQQRYDEIVVELVDRGYKINVDAKLDPDNVMTGEWYNDYTPTEDAFNIIRAQIAEKIAMKPSFYRFTKAKTSNN

SEQ ID NO: 11 - protein, 8 aa, Artificial sequence
  Synthetic peptide nuclear localization signal
PKKRKRRL

SEQ ID NO: 12 - protein, 8 aa, Artificial sequence
  Synthetic peptide nuclear localization signal
PKKKRKRL

SEQ ID NO: 13 - protein, 18 aa, Artificial sequence
  Synthetic peptide nuclear localization signal
MALHSMRKARERWSFIRA

SEQ ID NO: 14 - protein, 35 aa, Artificial sequence
  Synthetic peptide nuclear localization signal
MGVFCLGFWGLGRKLRTFGKGPKQLLSRLCGDHLQ

SEQ ID NO: 15 - protein, 29 aa, Artificial sequence
  Synthetic peptide nuclear localization signal
MGVFCLGFWGLGRKLRTFGKGPLQLLSRL

SEQ ID NO: 16 - DNA, 30 bp, Artificial sequence
  Synthetic oligonucleotide primer
cttgcccatatgacacgtgtgaatctcgta

SEQ ID NO: 17 - DNA, 30 bp, Artificial sequence
  Synthetic oligonucleotide primer
cttgcccatatgggcgtgttttgcttaggc

SEQ ID NO: 18 - DNA, 36 bp, Artificial sequence
  Synthetic oligonucleotide primer
cagttattatcgcgcttaacacgtgtgaatctcgta

SEQ ID NO: 19 - DNA, 39 bp, Artificial sequence
  Synthetic oligonucleotide primer
ccaaagaagaggaaaaggaggctaggatccatcgccacc

SEQ ID NO: 20 - DNA, 39 bp, Artificial sequence
  Synthetic oligonucleotide primer
ccaaagaagaggaaaaggaggctaaagctttatggccgc

SEQ ID NO: 21 - DNA, 36 bp, Artificial sequence
  Synthetic oligonucleotide primer
gcggccgctaagcttattattgctggttttagcttt

SEQ ID NO: 22 - DNA, 36 bp, Artificial sequence
  Synthetic oligonucleotide primer
gcggccgctaagctttagcctcttcctctttttctt

SEQ ID NO: 23 - DNA, 33 bp, Artificial sequence
  Synthetic peptide nuclear localization signal
gcggccgctaagcttgtacagctcgtccatgcc

SEQ ID NO: 24 - DNA, 36 bp, Artificial sequence
  Synthetic oligonucleotide primer
tacgagattcacacgtgttaagcgcgataataactg

SEQ ID NO: 25 - DNA, 39 bp, Artificial sequence
  Synthetic oligonucleotide primer
tagcctccttttcctcttctttggcccgggattattgct

SEQ ID NO: 26 - DNA, 426 bp, Chlorella virus-1
atgacacgtgtgaatctcgtaccggttcaagaattagctgaccagcatctcatggcagaatttcgtgaacttaagatgattccgaaggcactcgcaagaagtcttcgaactcaatcgtccgaaaaaatattgaagaagatcccatcaaaatttactctgaacactggtcatgttctgttcttttacgataaaggcaagtatttgcaacaacgatacgacgaaattgtcgttgaacttgttgatagggggtataagataaacgttgacgctaaactcgaccctgataacgtgatgacgggagagtggtacaatgattacaccccaacagaagatgcgtttaatattattcgagcgaggattgccgaaaaaatcgctatgaagccaagtttttacaggttcacgaaagctaaaaccagcaataattaa

SEQ ID NO: 27 - DNA, 417 bp, Bacteriophage T4
atgactcgtatcaaccttactttagtatctgaattggctgaccaacacttaatggctgaatatcgtgaattgccgcgtgtttttggtgcagttcgtaagcatgttgctaacggtaaacgtgttcgtgattttaaaatcagtcctacttttatccttggcgcaggtcatgttacattcttttacgataagctcgagttcttacgtaaacgtcaaattgagcttatagctgaatgtttaaaacgtggttttaatatcaaggatactacagtccaggatattagtgatattcctcaggaattccgtggtgattatattccccatgaagcttctattgctatatcacaagctcgtttagatgaaaaaattgcacaacgtcctacttggtacaaatactacggtaaggcgatttatgcataa

SEQ ID NO: 28 - protein, 8 aa, Artificial sequence
  Synthetic peptide nuclear localization signal
PKKKRKVC

SEQ ID NO: 29 - protein, 9 aa, Artificial sequence
  Synthetic peptide nuclear localization signal
GKKKYKLKH

SEQ ID NO: 30 - protein, 7 aa, Artificial sequence
  Synthetic peptide nuclear localization signal
KSKKKAQ

SEQ ID NO: 31 - protein, 10 aa, Artificial sequence
  Synthetic peptide nuclear localization signal
KELKQKQITK

SEQ ID NO: 32 - protein, 19 aa, Artificial sequence
  Synthetic peptide nuclear localization signal
NEWTLELLEELKNEAVRHF

SEQ ID NO: 33 - protein, 20 aa, Artificial sequence
  Synthetic peptide nuclear localization signal
RHSRIGVTRGRRARNGASRS

SEQ ID NO: 34 - protein, 12 aa, Artificial sequence
  Synthetic peptide nuclear localization signal
RQARRNRRRRWR

SEQ ID NO: 35 - protein, 7 aa, Artificial sequence
  Synthetic peptide nuclear localization signal
GKKRSKV

SEQ ID NO: 36 - protein, 7 aa, Artificial sequence
  Synthetic peptide nuclear localization signal
KSRKRKL

SEQ ID NO: 37 - protein, 18 aa, Artificial sequence
  Synthetic peptide nuclear localization signal
RPAATKKAGQAKKKKLDK

SEQ ID NO: 38 - protein, 20 aa, Artificial sequence
  Synthetic peptide nuclear localization signal
RKKRKTEEESPLKDKAKKSK

SEQ ID NO: 39 - protein, 20 aa, Artificial sequence
  Synthetic peptide nuclear localization signal
KKYENVVIKRSPRKRGRPRK

SEQ ID NO: 40 - protein, 11 aa, Human immunodeficiency virus
YGRKKRRQRRR

SEQ ID NO: 41 - protein, 9 aa, Human immunodeficiency virus
RKKRRQRRR

SEQ ID NO: 42 - protein, 9 aa, Human immunodeficiency virus
KKKKKKKKK

SEQ ID NO: 43 - protein, 9 aa, Human immunodeficiency virus
RRRRRRRRR